(19) United States
(12) Patent Application Publication
(10) Pub. No.: US 2006/0120956 A1
(43) Pub. Date: Jun. 8, 2006

(54) Imaging agents comprising barbituric acid derivatives

(76) Inventors: Klaus Kopka, Munster (DE); Hans-Jorg Breyholz, Munster (DE); Stefan Wagner, Munster (DE); Michael Shafers, Munster (DE); Bodo Leykau, Munster (DE); Benedicte Guilbert, Buckinghamshire (GB); Duncan Wynn, Buckinhamshire (GB)

Correspondence Address: GE HEALTHCARE, INC., IP DEPARTMENT, 101 CARNEGIE CENTER, PRINCETON, NJ, 08540-6231, US

(21) Appl. No.: 10/530,836

(22) PCT Filed: Oct. 8, 2003
(86) PCT No.: PCT/GB03/04351

§ 371 Date: Nov. 10, 2005

(30) Foreign Application Priority Data
Oct. 8, 2002 (GB) 0223249.4

(51) Int. Cl. A61K 51/00; C07F 5/00; C07D 403/02; C07K 16/46

(52) U.S. Cl. 424/1.11; 436/518; 530/391.1; 534/11; 544/295; 544/310

(57) ABSTRACT

The present invention relates to diagnostic imaging agents for in vivo imaging. The imaging agents comprise a synthetic barbituric acid derivative labelled at the 5-position with an imaging moiety suitable for diagnostic imaging in vivo. The invention also provides pharmaceutical and radiopharmaceutical compositions comprising the imaging agents, together with kits of the preparation of the radiopharmaceuticals. Also described are chelator conjugates of the barbituric acid derivative, which are suitable for the preparation of imaging agents comprising a radioactive or paramagnetic metal ion. The imaging agents are useful for the diagnostic imaging in vivo of various disease states, including atherosclerosis.

### FIELD OF THE INVENTION

The present invention relates to diagnostic imaging agents for in vivo imaging. The imaging agents comprise a synthetic barbituric acid derivative labelled at the 5-position with an imaging moiety suitable for diagnostic imaging in vivo.

### BACKGROUND TO THE INVENTION

Barbituric acid, or pyrimidine-2,4,6-trione is a known drug. Derivatives thereof, 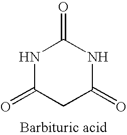
especially those arising from the introduction of substituents at the 5-position (ie. the CH₂ of the pyrimidine ring) are also known drugs. An example is barbital, ie. 5,5-diethylbarbituric acid.

Grigsby et al [J. Nucl. Med., 22(6), Abstract P12 (1981)] disclose lipophilic ⁷⁵Se and ^{123m}Te-labelled barbiturate derivatives, where the radioisotope is part of an aralkyl substituent at the 5-position, as potential regional cerebral blood flow imaging radiopharmaceuticals.

U.S. Pat. No. 3,952,091 discloses compounds useful in the in vitro radioimmunoassay of barbiturate drugs, which comprise barbituric acid labelled at the 5-position with the radioisotope. ¹²⁵I.

U.S. Pat. No. 4,244,939 discloses compounds useful in the in vitro radioimmunoassay of barbiturate drugs, which comprise barbituric acid labelled at 1- or 3-position (ie. the ring nitrogens), optionally via a linker group, with the radioisotopes ¹²⁵I or ¹³¹I.

WO 01/60416 discloses chelator conjugates of matrix metalloproteinase (MMP) inhibitors, and their use in the preparation of metal complexes with diagnostic metals. The specific classes of MMP inhibitor described are hydroxamates, especially succinyl hydroxamates.

### THE PRESENT INVENTION

It has now been found that synthetic barbituric acid matrix metalloproteinase (MMP) inhibitors labelled at the 5-position with an imaging moiety are useful diagnostic imaging agents for in vivo imaging of the mammalian body. Barbituric acid MMP inhibitors (ie. pyrimidine-2,4,6-triones) can exhibit greater selectivity than hydroxamic acid derivatives for selected MMPs, especially for the gelatinases (MMP-2 and MMP-9), and the membrane-bound MT-MMPs 1 (MMP-14) and 3 (MMP-16), plus MMP-8. For an imaging agent this results in decreased unwanted background activity, and hence improved signal to noise. Barbituric acid derivatives are also more lipophilic than hydroxamic acid or peptide-based MMP inhibitors, which means that the imaging agents of the present invention are better able to cross cell membranes or the blood-brain barrier due to their lipophilicity. Hence, the agents of the present invention are expected to be useful also for imaging brain disease such as brain tumours, amyotrophic lateral sclerosis, Alzheimer's disease or other sites of MMP activity within the brain.

The imaging agents of the present invention are useful for the in vivo diagnostic imaging of a range of disease states (inflammatory, malignant and degenerative diseases) where specific matrix metalloproteinases are known to be involved. These include:
(a) atherosclerosis, where various MMPs are overexpressed. Elevated levels of MMP-1, 3, 7, 9, 11, 12, 13 and MT1-MMP have been detected in human atherosclerotic plaques [S. J. George, Exp. Opin. Invest. Drugs, 9(5), 993-1007 (2000) and references therein]. Expression of MMP-2 [Z. Li et al, Am. J. Pathol., 148, 121-128 (1996)] and MMP-8 [M. P. Herman et al, Circulation, 104, 1899-1904 (2001)] in human atheroma has also been reported;
(b) chronic heart failure (Peterson, J. T. et al. Matrix metalloproteinase inhibitor development for the treatment of heart failure, Drug Dev. Res. (2002), 55(1), 29-44 reports that MMP-1, MMP-2, MMP-3, MMP-8, MMP-9, MMP-13 and MMP-14 are upregulated in heart failure);
(c) cancer [Vihinen et al, Int. J. Cancer 99, p157-166 (2002) reviews MMP involvement in cancers, and particularly highlights MMP-2, MMP-3, MMP-7, and MMP-9];
(d) arthritis [Jacson et al, Inflamnm. Res. 50(4), p183-186 (2001) “Selective matrix metalloproteinase inhibition in rheumatoid arthritis—targeting gelatinase A activation”, MMP-2 is particularly discussed];
(e) amyotrophic lateral sclerosis [Lim et al, J. Neurochem, 67, 251-259 (1996);
where MMP-2 and MMP-9 are involved];
(f) brain metastases, where MMP-2, MMP-9 and MMP-13 have been reported to be implicated [Spinale, Circul. Res., 90, 520-530 (2002)];
(g) cerebrovascular diseases, where MMP-2 and MMP-9 have been reported to be involved [Lukes et al, Mol. Neurobiol., 19, 267-284 (1999)];
(h) Alzheimer's disease, where MMP-2 and MMP-9 have been identified in diseased tissue [Backstrom et al, J. Neurochem., 58, 983-992 (1992)];
(i) neuroinflammatory disease, where MMP-2, MMP-3 and MMP-9 are involved [Mun-Bryce et al, Brain. Res., 933, 42-49 (2002)];
(j) COPD (ie. chronic obstructive pulmonary disease) where MMP-1, MMP-2, MMP-8 and MMP-9 have been reported to be upregulated [Segura-Valdez et al, Chest, 117, 684-694 (2000)];
(k) eye pathology [urpakus-Wheater et al, Prog. Histo. Cytochem., 36(3), 179-259 (2001)];
(l) skin diseases [Herouy, Y., Int. J. Mol. Med., 7(1), 3-12 (2001)].

### DETAILED DESCRIPTION OF THE INVENTION

In a first aspect, the present invention provides an imaging agent which comprises a synthetic barbituric acid matrix metalloproteinase inhibitor labelled at the 5-position of the barbituric acid with an imaging moiety, wherein the imaging moiety can be detected following administration of said labelled synthetic barbituric acid matrix metalloproteinase inhibitor to the mammalian body in vivo, and said imaging moiety is chosen from:

(i) a radioactive metal ion;

(ii) a paramagnetic metal ion;

(iii) a gamma-emitting radioactive halogen;

(iv) a positron-emitting radioactive non-metal;

(v) a hyperpolarised NMR-active nucleus;

(vi) a reporter suitable for in vivo optical imaging;

(vii) a β-emitter suitable for intravascular detection.

The synthetic barbituric acid matrix metalloproteinase inhibitor is suitably of molecular weight 100 to 2000 Daltons, preferably of molecular weight 150 to 600 Daltons, and most preferably of molecular weight 200 to 500 Daltons.

The imaging moiety may be detected either external to the mammalian body or via use of detectors designed for use in vivo, such as intravascular radiation or optical detectors such as endoscopes, or radiation detectors designed for intra-operative use. Preferred imaging moieties are those which can be detected externally in a non-invasive manner following administration in vivo. Most preferred imaging moieties are radioactive, especially radioactive metal ions, gamma-emitting radioactive halogens and positron-emitting radioactive non-metals, particularly those suitable for imaging using SPECT or PET.

When the imaging moiety is a radioactive metal ion, ie. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N and ¹⁸F, especially ¹¹C and ¹⁸F, most especially ¹⁸F.

When the imaging moiety is a hyperpolarised NMR-active nucleus, such NMR-active nuclei have a non-zero nuclear spin, and include ¹³C, ¹⁵N, ¹⁹F, ²⁹Si and ³¹P. Of these, ¹³C is preferred. By the term “hyperpolarised” is meant enhancement of the degree of polarisation of the NMR-active nucleus over its' equilibrium polarisation. The natural abundance of ¹³C (relative to ¹²C) is about 1%, and suitable ¹³C-labelled compounds are suitably enriched to an abundance of at least 5%, preferably at least 50%, most preferably at least 90% before being hyperpolarised. At least one carbon atom of a carbon-containing substituent at the 5-position of the barbituric acid of the present invention is suitably enriched with ¹³C, which is subsequently hyperpolarised.

When the imaging moiety is a reporter suitable for in vivo optical imaging, the reporter is any moiety capable of detection either directly or indirectly in an optical imaging procedure. The reporter might be a light scatterer (eg. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties.

Preferred organic chromophoric and fluorophoric reporters include groups having an extensive delocalized electron system, eg. cyanines, merocyanines, indocyanines, phthalocyanines, naphthalocyanines, triphenylmethines, porphyrins, pyrilium dyes, thiapyriliup dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly preferred are dyes which have absorption maxima in the visible or near infrared region, between 400 nm and 3 μm, particularly between 600 and 1300 nm.

Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

When the imaging moiety is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³ Br.

The imaging agents of the present invention are preferably of Formula I:
[{inhibitor}-(A)ₙ]ₘ-[imaging moiety] (I)
where:
{inhibitor} is the synthetic barbituric acid matrix metalloproteinase inhibitor;
-(A)ₙ— is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid or a monodisperse polyethyleneglycol (PEG) building block;
   where R is independently chosen from H, C₁₋₄ alkyl, C₂₋₄ alkenyl,
   C₂₋₄ alkynyl, C₁₋₄ alkoxyalkyl or C₁₋₄ hydroxyalkyl;
n is an integer of value 0 to 10, and
m is 1, 2 or 3.

It is envisaged that the role of the linker group -(A)ₙ— of Formula I is to distance the imaging moiety from the active site of the barbiturate metalloproteinase inhibitor. This is particularly important when the imaging moiety is relatively bulky (eg. a metal complex), so that binding of the inhibitor to the MMP enzyme is not impaired. This can be achieved by a combination of flexibility (eg. simple-alkyl chains), so that the bulky group has the freedom to position itself away from the active site and/or rigidity such as a cycloalkyl or aryl spacer which orientates the metal complex away from the active site.

The nature of the linker group can also be used to modify the biodistribution of the imaging agent. Thus, eg. the introduction of ether groups in the linker will help to minimise plasma protein binding. When -(A)ₙ- comprises a monodisperse polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

When -(A)ₙ- comprises a peptide chain of 1 to 10 amino acid residues, the amino acid residues are preferably chosen from glycine, lysine, aspartic acid or serine. When -(A)ₙ- comprises a PEG moiety, it preferably comprises a unit derived from polymerisation of the monodisperse PEG-like structure, 17-amino-5-oxo-6-aza-3, 9, 12, 15-tetraoxaheptadecanoic acid of Formula II: 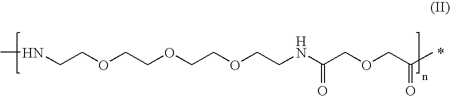
wherein n equals an integer from 1 to 10 and where the C-terminal unit (*) is connected to the imaging moiety.

When the linker group does not comprise PEG or a peptide chain, preferred -(A)ₙ- groups have a backbone chain of linked atoms which make up the -(A)ₙ- moiety of 2 to 10 atoms, most preferably 2 to 5 atoms, with 2 or 3 atoms being especially preferred. A minimum linker group backbone chain of 2 atoms confers the advantage that the imaging moiety is well-separated from the barbituric acid metalloproteinase inhibitor so that any interaction is minimised.

Non-peptide linker groups such as alkylene groups or arylene groups have the advantage that there are no significant hydrogen bonding interactions with the conjugated barbituric acid MMP inhibitor, so that the linker does not wrap round onto the barbituric acid MMP inhibitor. Preferred alkylene spacer groups are —(CH₂)_{q}— where q is 2 to 5. Preferred arylene spacers are of formula:

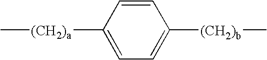

where: a and b are independently 0, 1 or 2.

The linker group -(A)ₙ- is preferably derived from glutaric acid, succinic acid, a polyethyleneglycol based unit or a PEG-like unit of Formula II.

When the imaging moiety comprises a metal ion, the metal ion is present as a metal complex. Such barbituric acid metalloproteinase inhibitor conjugates with metal ions are therefore suitably of Formula Ia:
[{inhibitor}-(A)ₙ]ₘ-[metal complex] (Ia)

where: A, n and m are as defined for Formula I above.

By the term “metal complex” is meant a coordination complex of the metal ion with one or more ligands. It is strongly preferred that the metal complex is “resistant to transchelation”, ie. does not readily undergo ligand exchange with other potentially competing ligands for the metal coordination sites. Potentially competing ligands include the barbituric acid moiety itself plus other excipients in the preparation in vitro (eg. radioprotectants or antimicrobial preservatives used in the preparation), or endogenous compounds in vivo (eg. glutathione, transferrin or plasma proteins).

The metal complexes of Formula I are derived from conjugates of ligands of Formula Ib:
[{inhibitor}-(A)ₙ]ₘ-[ligand] (Ib)

In Formulae I, Ia and Ib, m is preferably 1 or 2, and is most preferably 1.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium which form metal complexes resistant to transchelation include, but are not limited to:
(i) diaminedioximes of formula: 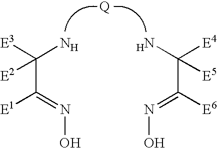
where E¹-E⁶ are each independently an R′ group;
each R′ is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R′ groups together with the atoms to which they are attached form a carbocyclic, heterocyclic, saturated or unsaturated ring, and wherein one or more of the R′ groups is conjugated to the barbituric acid MMP inhibitor;
and Q is a bridging group of formula -(J)_{f}-;
where f is 3, 4 or 5 and each J is independently —O—, —NR′— or —C(R′)₂— provided that -(J)_{f}- contains a maximum of one J group which is —O— or —NR′—.

Preferred Q groups are as follows:

Q=—(CH₂)(CHR′)(CH₂)— ie. propyleneamine oxime or PnAO derivatives;

Q=—(CH₂)₂(CHR′)(CH₂)₂— ie. pentyleneamine oxime or PentAO derivatives;

Q=—(CH₂)₂NR′(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The barbituric acid MMP inhibitor is preferably conjugated at either the E¹ or E⁶ R′ group, or an R′ group of the Q moiety. Most preferably, the barbituric acid MMP inhibitor is conjugated to an R′ group of the Q moiety. When the barbituric acid MMP inhibitor is conjugated to an R′ group of the Q moiety, the R′ group is preferably at the bridgehead position. In that case, Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—. An especially preferred bifunctional diaminedioxime chelator has the Formula m (Chelator 1): 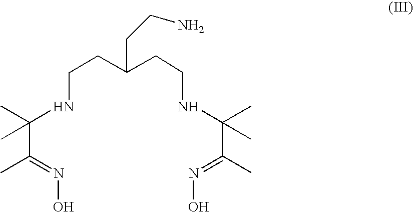
such that the synthetic barbituric acid MMP inhibitor is conjugated via the bridgehead —CH₂CH₂NH₂ group.
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam.
(v) N₂O₂ ligands having a diaminediphenol donor set.

The above described ligands are particularly suitable for complexing technetium eg. ^{94m}Tc or ^{99m}Tc, and are described more fully by Jurisson et al [Chem. Rev., 99, 2205-2218 (1999)]. The ligands are also useful for other metals, such as copper (⁶⁴Cu or ⁶⁷C), vanadium (eg. ⁴⁸V), iron (eg. ⁵²Fe), or cobalt (eg. ⁵⁵Co). Other suitable ligands are described in Sandoz WO 91/01144, which includes ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. When the radiometal ion is technetium, the ligand is preferably a chelating agent which is tetradentate. Preferred chelating agents for technetium are the diaminedioximes, or those having an N₂S₂ or N₃S donor set as described above. Especially preferred chelating agents for technetium are the diaminedioximes.

It is strongly preferred that the synthetic barbituric acid matrix metalloproteinase inhibitor is bound to the metal complex in such a way that the linkage does not undergo facile metabolism in blood, since that would result in the metal complex being cleaved off before the labelled metalloproteinase inhibitor reached the desired in vivo target site. The synthetic barbituric acid matrix metalloproteinase inhibitor is therefore preferably covalently bound to the metal complexes of the present invention via linkages which are not readily metabolised.

When the imaging moiety is a radioactive halogen, such as iodine, the barbituric acid MMP inhibitor is suitably chosen to include: a non-radioactive halogen atom such as an aryl iodide or bromide (to permit radioiodine exchange); an activated aryl ring (e.g. a phenol group); an organometallic precursor compound (eg. trialkyltin or trialkylsilyl); or an organic precursor such as triazenes. Methods of introducing radioactive halogens (including ¹²³I and ¹⁸F) are described by Bolton [J. Lab. Comp. Radiopharm., 45, 485-528 (2002)]. Examples of suitable aryl groups to which radioactive halogens, especially iodine can be attached are given below:

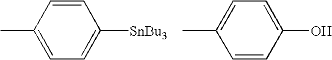

Both contain substituents which permit facile radioiodine substitution onto the aromatic ring. Alternative substituents containing radioactive iodine can be synthesised by direct iodination via radiohalogen exchange, e.g.

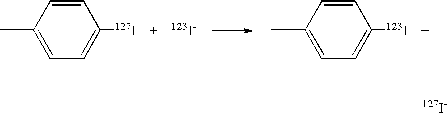

When the imaging moiety is a radioactive isotope of iodine the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

When the imaging moiety comprises a radioactive isotope of fluorine (eg. ¹⁸F), the radioiodine atom may be carried out via direct labelling using the reaction of ¹⁸F-fluoride with a suitable precursor having a good leaving group, such as an alkyl bromide, alkyl mesylate or alkyl tosylate. ¹⁸F can also be introduced by N-alkylation of amine precursors with alkylating agents such as ¹⁸F(CH₂)₃OMs (where Ms is mesylate) to give N—(CH₂)₃¹⁸F, or O-alkylation of hydroxyl groups with ¹⁸F(CH₂)₃OMs or ¹⁸F(CH₂)₃Br. For aryl systems, ¹⁸F-fluoride displacement of nitrogen from an aryl diazonium salt is a good route to aryl-¹⁸F derivatives. See Bolton, J. Lab. Comp. Radiopharm., 45, 485-528 (2002) for a description of routes to ¹⁸F-labelled derivatives.

Preferred synthetic barbituric acid matrix metalloproteinase inhibitors of the present invention are of Formula IV: 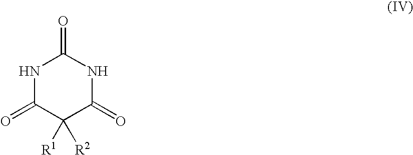
where:
R¹ is R″ or a Z group;
R² is R″, Y or —NR⁴R⁵, where R⁴ is H or an R″ group, R⁵ is H, C₂₋₁₄ acyl, C₂₋₁₀ aminoalkyl or (N—C₂₋₁₄ acyl)C₂₋₁₀ aminoalkyl or an R″ group, or R⁴ and R⁵ together with the N atom to which they are attached form an optionally (N—C₂₋₁₄)acylated C₂₋₈ cycloaminoalkylene ring;
R″ is independently C₁₋₁₄ alkyl, C₃₋₈ cycloalkyl, C₂₋₁₄ alkenyl, C₁₋₁₄ fluoroalkyl, C₁₋₁₄ perfluoroalkyl, C₆₋₁₄ aryl, C₂₋₁₄ heteroaryl or C₇₋₁₆ alkylaryl;
Z is a group of formula -A¹O[A²O]ₚR³ where p is 0 or 1, and A¹ and A² are independently C₁₋₁₀ alkylene, C₃₋₈ cycloalkylene, C₁₋₁₀ perfluoroalkylene, C₆₋₁₀ arylene or C₂₋₁₀ heteroarylene, and R³ is an R group where R is independently chosen from H, C₁₋₄ alkyl, C₂₋₄ alkenyl, C₂₋₄ allynyl, C₁₋₄ alkoxyalkyl or C₁₋₄ hydroxyalkyl;
Y is a group of formula:

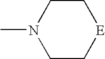

where E is CR₂, O, S or NR⁶; and R⁶ is C₂₋₁₄ acyl or an R″ or Z group.

In Formula IV, R² is preferably Y or —NR⁴R⁵. When the imaging agent comprises a barbituric acid MMP inhibitor of Formula IV, and the imaging moiety is a gamma-emitting radioactive halogen or a positron-emitting radioactive non-metal, the imaging moiety may be attached at either of the R¹ or R² substituents. When the imaging moiety is a radioactive or paramagnetic metal ion, the R² substituent of Formula IV is preferably attached to or comprises the imaging moiety.

Especially preferred synthetic barbituric acid matrix metalloproteinase inhibitors of the present invention are of Formula V: 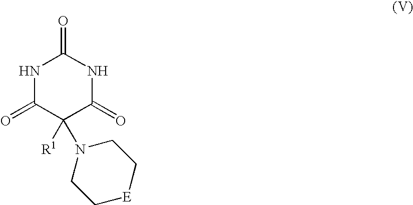
where E is CHR or NR⁶ and R¹ is C₆₋₁₄ n-alkyl, or C₆₋₁₄ aryl. Preferred synthetic barbituric acid matrix metalloproteinase inhibitors of Formula V are those having E=NR⁶ and R⁶═C₂₋₁₄ acyl; —(CH₂)_{d}OH, where d is 2, 3, 4 or 5; or —C₆H₄ where X is H, C₁₋₄ alkyl, Hal, OR, NR₂, NO₂ or SO₂NR⁷R⁸, where R⁷ and R⁸ are independently R groups, and R is as defined in Formula IV (above).

Especially preferred synthetic barbituric acid matrix metalloproteinase inhibitors of Formula V are those where R¹ is n-octyl, n-decyl, biphenyl, C₆H₅X or —C₆H₄—O—C₆H₄X where X is as defined above.

The barbituric acid MMP inhibitor compounds of the present invention are prepared by condensation of urea with mono- or di-substituted malonic ester derivatives. Further details are described by Foley et al [Bioorg. Med. Chem. Lett, 11, 969-972 (2001)]. The MMP inhibitor compounds of Formula V can be prepared by the method of Grams et al [Biol. Chem., 382, 1277-1285 (2001)].

When the imaging agent of the present invention comprises a radioactive or paramagnetic metal ion, the metal ion is suitably present as a metal complex. Such metal complexes are suitably prepared by reaction of the conjugate of Formula Ib with the appropriate metal ion. The ligand-conjugate or chelator-conjugate of the barbituric acid MMP inhibitor of Formula Ib can be prepared via the bifunctional chelate approach. Thus, it is well known to prepare ligands or chelating agents which have attached thereto a functional group (“bifunctional linkers” or “bifunctional chelates” respectively). Functional groups that have been attached include: amine, thiocyanate, maleimide and active esters such as N-hydroxysuccinimide or pentafluorophenol. Chelator 1 of the present invention is an example of an amine-functionalised bifunctional chelate. Such bifunctional chelates can be reacted with suitable functional groups on the barbituric acid matrix metalloproteinase inhibitor to form the desired conjugate. Such suitable functional groups on the barbituric acid include:

carboxyls (for amide bond formation with an amine-functionalised bifunctional chelator);

amines (for amide bond formation with an carboxyl- or active ester-functionalised bifunctional chelator);

halogens, mesylates and tosylates (for N-alkylation of an amine-functionalised bifunctional chelator) and

thiols (for reaction with a maleimide-functionalised bifunctional chelator).

The radiolabelling of the especially preferred barbiturate MB inhibitors of the present invention can be conveniently carried out using “precursors”. When the imaging moiety comprises a metal ion, such precursors suitably comprise “conjugates” of the barbiturate MMP inhibitor with a ligand, as described in the fourth embodiment below. When the imaging moiety comprises a non-metallic radioisotope, ie. a gamma-emitting radioactive halogen or a positron-emitting radioactive non-metal, such “precursors” suitably comprise a non-radioactive material which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors can conveniently be obtained in good chemical purity and, optionally supplied in sterile form.

It is envisaged that “precursors” (including ligand conjugates) for radiolabelling of the especially preferred barbiturate MMP inhibitors of the present invention can be prepared as follows:

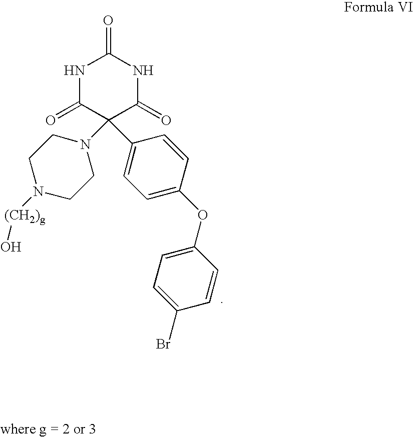

The terminal —OH group of the compound of Formula VI may be converted to a tosyl or mesyl group or bromo derivative, which can then be used to conjugate an amino-functionalised chelator (shown in Scheme 1 for g=2):

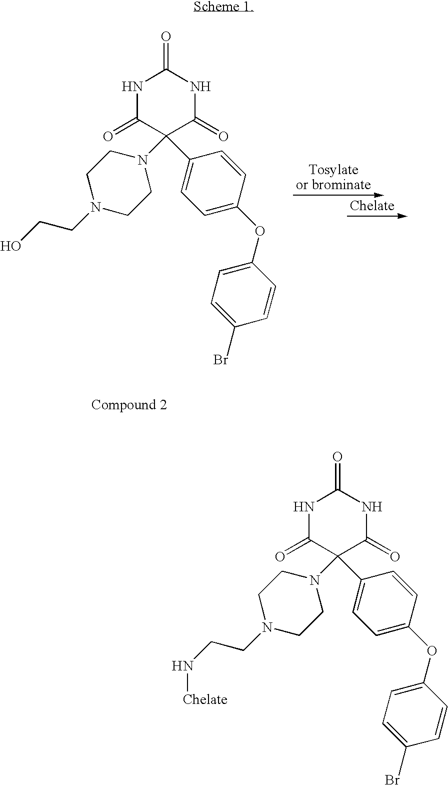

The tosylate, mesylate or bromo groups of the precursors described may alternatively be displaced with [¹⁸F]fluoride to give an ¹⁸F-labelled PET imaging agent.

Radioiodine derivatives can be prepared from the corresponding phenol precursors:

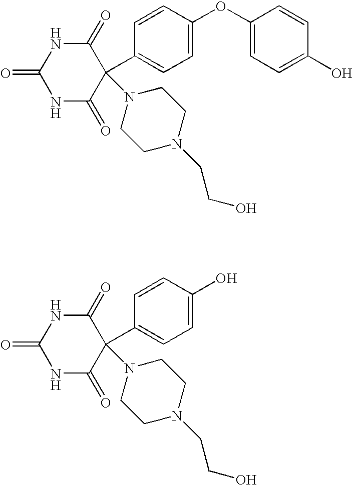

An alternative approach would be to use Compound 23 [Grams et al, Biol. Chem., 382, 1277-1285 (2001) and Example 5 step (h)] for N-alkylation of an amine-functionalised chelator:

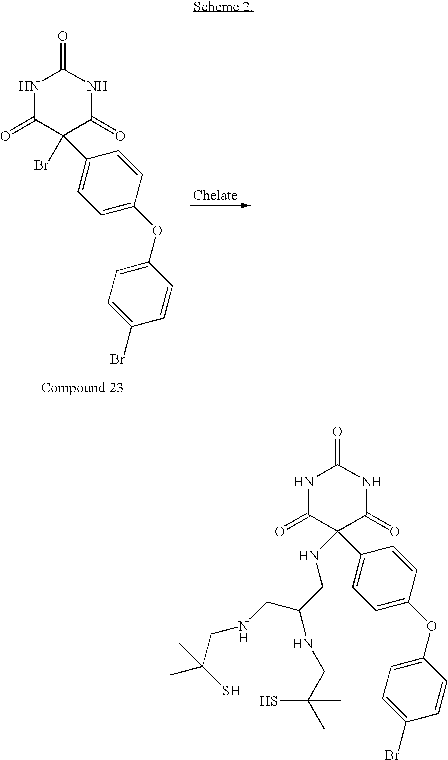

Compound 23 can also be reacted with amines to give precursors suitable for radioiodination, such as:

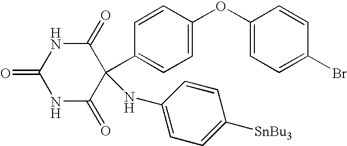

The non-radioactive iodinated analogue Compound 24 has been prepared:

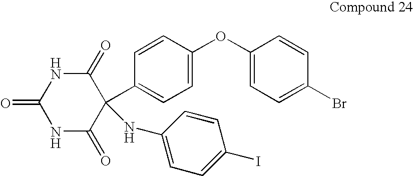

Compound 23 can also be converted to an aryl trimethylsilyl (TMS) precursor for radioiodination:

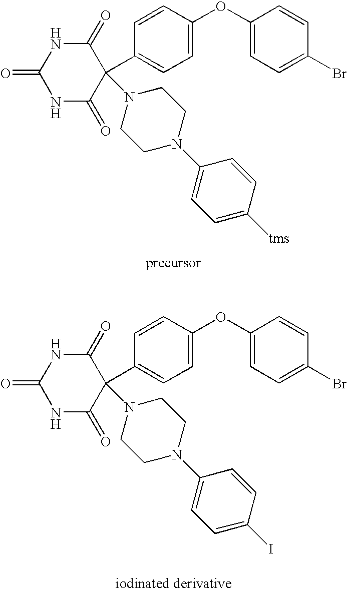

Compound 23 can be converted to an aryl diazonium precursor for radiofluorination as shown in Scheme 6:

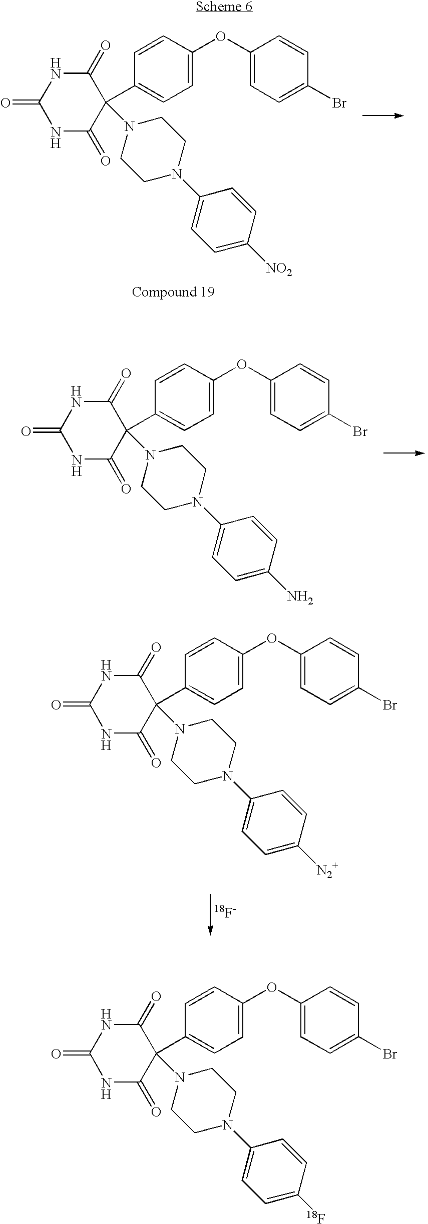

Another approach would be to employ an amino group at the C-5 position. In this way it is expected that a chelator could be conjugated via a linker (Scheme 3):

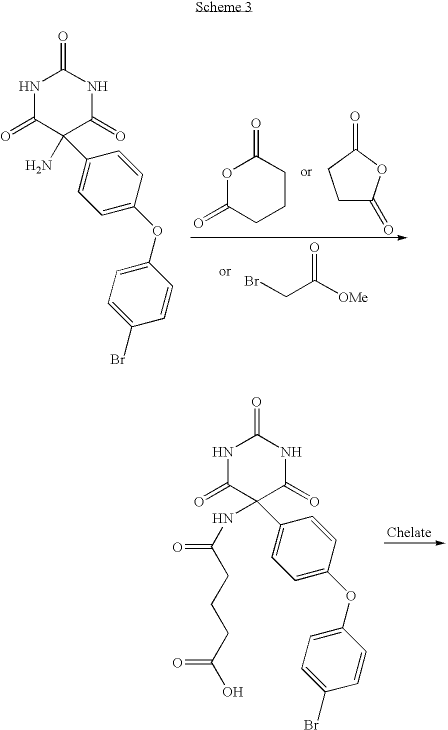

Such primary amine substituted barbiturates can be prepared by alkylation of Compound 23 with benzylamine, followed by removal of the benzyl protecting group under standard conditions such as hydrogenation using a palladium catalyst on charcoal.

Another approach would be to use the piperazine derivative (Compound 6, Example 7) to attach a chelate. This could be via direct conjugation of the piperazine substituent secondary amine with a carboxyl- or active ester-functionalised bifunctional chelator, or via a linker. The latter is illustrated in Scheme 4, where an amine-functionalised chelator would be attached to the pendant carboxyl function of the linker:

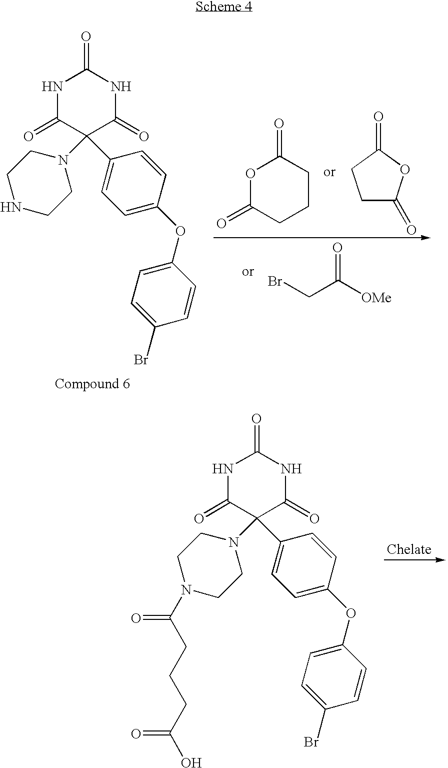

Compound 6 can be acylated to give precursors suitable for radioiodination:

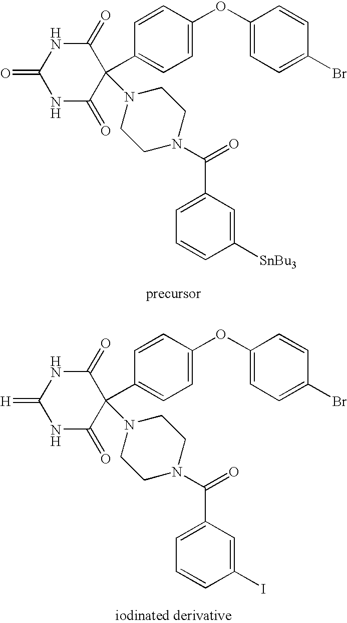

Compound 6 can also be reacted with a alkylating agent suitable for ¹⁸F labelling such as ¹⁸F(CH₂)₂OTs (where Ts is a tosylate group) or ¹⁸F(CH₂)₂OMs (where Ms is a mesylate group), to give the corresponding N-functionalised piperazine derivative having an N(CH₂)₂¹⁸F substituent. Alternatively, Compound 6 can first be reacted with chloroacetyl chloride to give the N(CO)CH₂Cl N-derivatised piperazine (Compound 11), followed by reaction with HS(CH₂)₃¹⁸F:

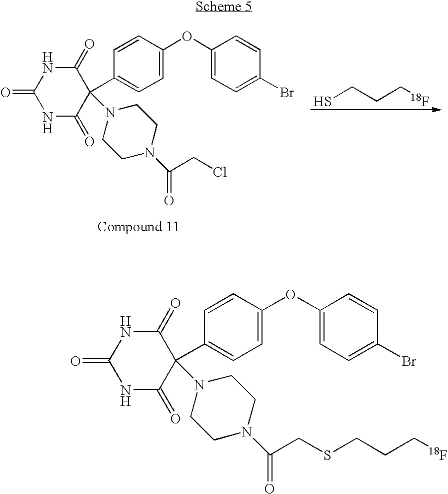

When the imaging agent of the present invention comprises a radioactive or paramagnetic metal ion, the metal ion is suitably present as a metal complex. Such metal complexes are suitably prepared by reaction of the conjugate of Formula Ib with the appropriate metal ion. The ligand-conjugate or chelator-conjugate of the barbituric acid MMP inhibitor of Formula Ib can be prepared via the bifunctional chelate approach. Thus, it is well known to prepare ligands or chelating agents which have attached thereto a functional group (“bifunctional linkers” or “bifunctional chelates” respectively). Functional groups that have been attached include: amine, thiocyanate, maleimide and active esters such as N-hydroxysuccinimide or pentafluorophenol. Chelator 1 of the present invention is an example of an amine-functionalised bifunctional chelate. Such bifunctional chelates can be reacted with suitable functional groups on the barbituric acid matrix metalloproteinase inhibitor to form the desired conjugate. Such suitable functional groups on the barbituric acid include:

carboxyls (for amide bond formation with an amine-functionalised bifunctional chelator);

amines (for amide bond formation with an carboxyl- or active ester-functionalised bifunctional chelator);

halogens, mesylates and tosylates (for N-alkylation of an amine-functionalised bifunctional chelator) and

thiols (for reaction with a maleimide-functionalised bifunctional chelator).

The radiometal complexes of the present invention may be prepared by reacting a solution of the radiometal in the appropriate oxidation state with the ligand conjugate of Formula Ia at the appropriate pH. The solution may preferably contain a ligand which complexes weakly to the metal (such as gluconate or citrate) i.e. the radiometal complex is prepared by ligand exchange or transchelation. Such conditions are useful to suppress undesirable side reactions such as hydrolysis of the metal ion. When the radiometal ion is ^{99m}Tc, the usual starting material is sodium pertechnetate from a ⁹⁹Mo generator. Technetium is present in ^{99m}Tc-pertechnetate in the Tc(VII) oxidation state, which is relatively unreactive. The preparation of technetium complexes of lower oxidation state Tc(I) to Tc(V) therefore usually requires the addition of a suitable pharmaceutically acceptable reducing agent such as sodium dithionite, sodium bisulphite, ascorbic acid, formamidine sulphinic acid, stannous ion, Fe(II) or Cu(I), to facilitate complexation. The pharmaceutically acceptable reducing agent is preferably a stannous salt, most preferably stannous chloride, stannous fluoride or stannous tartrate.

When the imaging moiety is a hyperpolarised NMR-active nucleus, such as a hyperpolarised ¹³C atom, the desired hyperpolarised compound can be prepared by polarisation exchange from a hyperpolarised gas (such as ¹²⁹Xe or ³He) to a suitable ¹³C-enriched barbituric acid derivative.

In a second aspect, the present invention provides a pharmaceutical composition which comprises the imaging agent as described above, together with a biocompatible carrier, in a form suitable for mammalian administration. The “biocompatible carrier” is a fluid, especially a liquid, which in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like).

In a third aspect, the present invention provides a radiopharmaceutical composition which comprises the imaging agent as described above wherein the imaging moiety is radioactive, together with a biocompatible carrier (as defined above), in a form suitable for mammalian administration. Such radiopharmaceuticals are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, and are therefore preferably a disposable or other syringe suitable for clinical use. The pre-filled syringe may optionally be provided with a syringe shield to protect the operator from radioactive dose. Suitable such radiopharmaceutical syringe shields are known in the art and preferably comprise either lead or tungsten.

When the imaging moiety comprises ^{99m}Tc, a radioactivity content suitable for a diagnostic imaging radiopharmaceutical is in the range 180 to 1500 MBq of ^{99m}Tc, depending on the site to be imaged in vivo, the uptake and the target to background ratio.

In a fourth aspect, the present invention provides a conjugate of a synthetic barbituric acid matrix metalloproteinase inhibitor with a ligand, wherein the barbituric acid comprises a 5-position substituent, and said 5-position substituent comprises a ligand. Said ligand conjugates are useful for the preparation of synthetic barbituric acid matrix metalloproteinase inhibitor labelled with either a radioactive metal ion or paramagnetic metal ion. Preferably, the ligand conjugate is of Formula Ib, as defined above. Most preferably, the synthetic barbituric acid MMP inhibitor of the ligand conjugate is of Formula IV, as defined above. Ideally, the synthetic barbituric acid MMP inhibitor of the ligand conjugate is of Formula V, as defined above. The ligand of the conjugate of the fourth aspect of the invention is preferably a chelating agent. Preferably, the chelating agent has a diaminedioxime, N₂S₂, or N₃S donor set.

In a fifth aspect, the present invention provides precursors useful in the preparation of radiopharmaceutical preparations where the imaging moiety comprises a non-metallic radioisotope, ie. a gamma-emitting radioactive halogen or a positron-emitting radioactive non-metal. Such “precursors” suitably comprise a non-radioactive derivative of the synthetic barbiturate matrix metalloproteinase inhibitor material which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors can conveniently be obtained in good chemical purity. Suitable precursor derivatives are described in general terms by Bolton, J. Lab. Comp. Radiopharm., 45, 485-528 (2002).

Preferred precursors of this embodiment comprise a derivative which either undergoes electrophilic or nucleophilic halogenation; undergoes facile alkylation with an alkylating agent chosen from an alkyl or fluoroalkyl halide, tosylate, triflate (ie. trifluoromethanesulphonate) or mesylate; or alkylates thiol moieties to form thioether linkages. Examples of the first category are:
(a) organometallic derivatives such as a trialkylstannane (eg. trimethylstannyl or tributylstannyl), or a trialkylsilane (eg. trimethylsilyl);
(b) alkyl or aryl iodides or bromides for halogen exchange, and alkyl tosylates or mesylates for nucleophilic halogenation;
(c) aromatic rings activated towards electrophilic halogenation (eg. phenols) and aromatic rings activated towards nucleophilic halogenation (eg. aryl iodonium, aryl diazonium or nitroaryl compounds).

Preferred derivatives which undergo facile alkylation are alcohols, phenols or amine groups, especially phenols and sterically-unhindered primary or secondary amines.

Preferred derivatives which alkylate thiol-containing radioisotope reactants are N-haloacetyl groups, especially N-chloroacetyl and N-bromoacetyl derivatives.

Preferred convenient chemical forms of the desired non-metallic radioisotope include:
(a) halide ions (eg. ¹²³I-iodide or ¹⁸F-fluoride), especially in aqueous media, for substitution reactions;
(b) ¹¹C-methyl iodide or ¹⁸F-fluoroalkylene compounds functionalised with a good leaving group, such as bromide, mesylate or tosylate;
(c) HS(CH₂)₃¹⁸F for S-alkylation reactions with alkylating precursors such as N-chloroacetyl or N-bromoacetyl derivatives.

Examples of suitable such “precursors”, and methods for their preparation are described in the first embodiment (above).

In a sixth aspect, the present invention provides a non-radioactive kit for the preparation of radioactive metal ion radiopharmaceutical compositions described above, which comprises a conjugate of a ligand with a synthetic barbituric acid matrix metalloproteinase inhibitor. The ligand conjugates, and preferred aspects thereof, are described in the fourth embodiment above. Such kits are designed to give sterile radiopharmaceutical products suitable for human administration, e.g. via direct injection into the bloodstream. The kit is preferably lyophilised and is designed to be reconstituted with a convenient sterile source of the radiometal [eg. ^{99m}Tc-pertechnetate (TcO₄⁻) from a ^{99m}Tc radioisotope generator], to give a solution suitable for human administration without further manipulation. Suitable kits comprise a container (eg. a septum-sealed vial) containing the ligand or chelator conjugate in either free base or acid salt form. Alternatively, the kit may optionally contain a metal complex which, upon addition of the radiometal, undergoes transmetallation (i.e. metal exchange) giving the desired product.

When the radioactive metal ion is ^{99m}Tc, the kit preferably further comprises a biocompatible reductant, such as sodium dithionite, sodium bisulphite, ascorbic acid, formamidine sulphinic acid, stannous ion, Fe(II) or Cu(I). The biocompatible reductant is preferably a stannous salt such as stannous chloride or stannous tartrate.

The non-radioactive kits may optionally further comprise additional components such as a transchelator, radioprotectant, antimicrobial preservative, pH-adjusting agent or filler.

The “tanschelator” is a compound which reacts rapidly to form a weak complex with the radiometal, then is displaced by the ligand of the “conjugate”. This minimises the risk of formation of radioactive impurities, eg. reduced hydrolysed technetium (RHT) due to rapid reduction of pertechnetate competing with technetium complexation. Suitable such transchelators are salts of a weak organic acid, ie. an organic acid having a pKa in the range 3 to 7, with a biocompatible cation. Suitable such weak organic acids are acetic acid, citric acid, tartaric acid, gluconic acid, glucoheptonic acid, benzoic acid, phenols or phosphonic acids. Hence, suitable salts are acetates, citrates, tartrates, gluconates, glucoheptonates, benzoates, phenolates or phosphonates. Preferred such salts are tartrates, gluconates, glucoheptonates, benzoates, or phosphonates, most preferably phosphonates, most especially diphosphonates. A preferred such transchelator is a salt of MDP, ie. methylenediphosphonic acid, with a biocompatible cation.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

In a seventh aspect, the present invention provides kits for the preparation of radiopharmaceutical preparations where the imaging moiety comprises a non-metallic radioisotope, ie. a gamma-emitting radioactive halogen or a positron-emitting radioactive non-metal. Such kits comprise the “precursor” of the fifth embodiment, preferably in sterile non-pyrogenic form, so that reaction with a sterile source of the radioisotope gives the desired radiopharmaceutical with the minimum number of manipulations. Such considerations are particularly important for radiopharmaceuticals where the radioisotope has a relatively short half-life, and for ease of handling and hence reduced radiation dose for the radiopharmacist. Hence, the reaction medium for reconstitution of such kits is preferably aqueous, and in a form suitable for mammalian administration.

The “precursor” of the kit is preferably supplied covalently attached to a solid support matrix. In that way, the desired radiopharmaceutical product forms in solution, whereas starting materials and impurities remain bound to the solid phase. Precursors for solid phase electrophilic fluorination with ¹⁸F-fluoride are described in WO 03/002489. Precursors for solid phase nucleophilic fluorination with ¹⁸F-fluoride are described in WO 03/002157. The kit may therefore contain a cartridge which can be plugged into a suitably adapted automated synthesizer. The cartridge may contain, apart from the solid support-bound precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customer requirements for radioactive concentration, volumes, time of delivery etc. Conveniently, all components of the kit are disposable to minimise the possibility of contamination between runs and will be sterile and quality assured.

In an eighth aspect, the present invention discloses the use of the synthetic barbituric acid matrix metalloproteinase inhibitor imaging agent described above for the diagnostic imaging of atherosclerosis, especially unstable vulnerable plaques.

In a further aspect, the present invention discloses the use of the synthetic barbituric acid matrix metalloproteinase inhibitor imaging agent described above for the diagnostic imaging of other inflammatory diseases, cancer, or degenerative diseases.

In a further aspect, the present invention discloses the use of the synthetic barbituric acid matrix metalloproteinase inhibitor imaging agent described above for the intravascular detection of atherosclerosis, especially unstable vulnerable plaques, using proximity detection. Such proximity detection may be achieved using intravascular devices such as catheters or intra-operatively using hand-held detectors (eg. gamma detectors). Such intravascular detection is particularly useful when the imaging moiety is a reporter group suitable for in vivo optical imaging or a remitter, since such moieties may not be readily detected outside the mammalian body, but are suitable for proximity detection.

The invention is illustrated by the non-limiting Examples detailed below. Example 1 describes the synthesis of the compound 1,1,1-tris(2-aminoethyl)methane. Example 2 provides an alternative synthesis of 1,1,1-tris(2-aminoethyl)methane which avoids the use of potentially hazardous azide intermediates. Example 3 describes the synthesis of a chloronitrosoalkane precursor. Example 4 describes the synthesis of a preferred amine-substituted bifunctional diaminedioxime of the present invention (Chelator 1). Example 5 provides the synthesis of a non-radioactive iodinated barbiturate (Compound 4). Example 6 describes the synthesis of the radioiodinated ¹²⁵I analogue of Compound 4 (Compound 5). Example 7 describes the synthesis of a piperazine-substituted barbiturate (Compound 6), where the piperazine amine can be used for further conjugation (eg. of chelating agents). Example 8 describes the synthesis of a fluoropropyl derivative (Compound 7), and Example 9 the corresponding ¹⁸F analogue. Example 10 provides a thioether-linked fluoropropyl derivative (Compound 9), and Example 11 the corresponding ¹⁸F derivative (Compound 10). Example 12 provides a synthesis of a chloroacetyl intermediate (Compound 11). Examples 13 and 14 provide the syntheses of chelator conjugates of the present invention (Compounds 16 and 17). Example 15 provides the synthesis of a tributylstannyl radioiodination precursor (Compound 18). Example 16 describes the synthesis of a bromoethyl derivative (Compound 13) that acts as a precursor for the radiosynthesis of the corresponding ¹⁸F analogue via fluorodebromination with [¹⁸F]fluoride. Example 17 provides the synthesis of various phenylpiperazine derivatives (Compounds 19 to 22). Example 18 describes the synthesis of Compound 24. Examples 19 and 20 describe in vitro assays for assessing the inhibitory activity of compounds of the invention vs specific metalloproteinase enzymes. Table 1 and Table 2 show the inhibition assay results for examples of non-radioactive iodinated, fluorinated and chelate derivatives of the invention with respect to MMP-2, MMP-9 and MMP-12. This shows that most compounds have similar inhibitory activity to that of the comparative prior art Compounds 2 and 3. This demonstrates that a chelator or an imaging moiety such as an iodine atom or a fluorine atom can be introduced without compromising the biological activity of the barbiturate MMP inhibitor. Example 21 describes the ^{99m}Tc-radiolabelling of chelator conjugates of the invention. Example 22 describes a general method of radioiodination of suitable non-radioactive precursors of the invention.

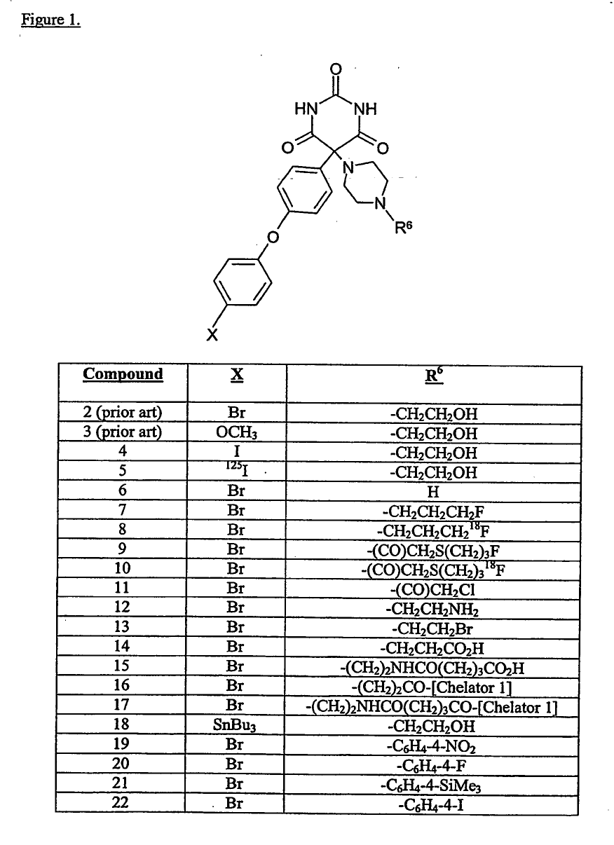

FIG. 1 shows the chemical structures of several compounds of the invention.

### EXAMPLE 1

### Synthesis of 1,1,1-tris(2-aminoethyl)methane

### Step 1(a): 3-(methoxycarbonylmethylene)glutaric acid dimethylester.

Carbomethoxymethylenetriphenylphosphorane (167 g, 0.5 mol) in toluene (600 ml) was treated with dimethyl 3-oxoglutarate (87 g, 0.5 mol) and the reaction heated to 100° C. on an oil bath at 120° C. under an atmosphere of nitrogen for 36 h. The reaction was then concentrated in vacuo and the oily residue triturated with 40/60 petrol ether/diethylether 1:1, 600 ml. Triphenylphosphine oxide precipitated out and the supernatant liquid was decanted/filtered off. The residue on evaporation in vacuo was Kugelrohr distilled under high vacuum Bpt (oven temperature 180-200° C. at 0.2 torr) to give 3-(methoxycarbonylmethylene)glutaric acid dimethylester (89.08 g, 53%).

NMR ¹H(CDCl₃): δ 3.31 (2H, s, CH₂), 3.7(9H, s, 3×OCH₃), 3.87 (2H, s, CH₂), 5.79 (1H, s, ═CH,) ppm.

NMR ¹³C(CDCl₃), δ 36.56, CH₃, 48.7, 2×CH₃, 52.09 and 52.5 (2×CH₂); 122.3 and 146.16 C═CH; 165.9, 170.0 and 170.5 3×COO ppm.

### Step 1(b): Hydrogenation of 3-(methoxycarbonylmethylene)glutaric acid dimethylester

3-(methoxycarbonylmethylene)glutaric acid dimethylester (89 g, 267 mmol) in methanol (200 ml) was shaken with (10% palladium on charcoal: 50% water) (9 g) under an atmosphere of hydrogen gas (3.5 bar) for (30 h). The solution was filtered through kieselguhr and concentrated in vacuo to give 3-(methoxycarbonylmethyl)glutaric acid dimethylester as an oil, yield (84.9 g, 94%).

NMR ¹H(CDCl₃), δ 2.48 (6H, d, J=8 Hz, 3×CH₂), 2.78 (1H, hextet, J=8 Hz CH,) 3.7 (9H, s, 3×CH₃).

NMR ¹³C(CDCl₃), δ 28.6, CH; 37.50, 3×CH₃; 51.6, 3×CH₂; 172.28,3×COO.

### Step 1(c): Reduction and Esterification of Trimethyl Ester to the Triacetate.

Under an atmosphere of nitrogen in a 3 necked 2L round bottomed flask lithium aluminium hydride (20 g, 588 mmol) in tetrahydrofuran (400 ml) was treated cautiously with tris(methyloxycarbonylmethyl)methane (40 g, 212 mmol) in tetrahydrofuran (200 ml) over 1 h. A strongly exothermic reaction occurred, causing the solvent to reflux strongly. The reaction was heated on an oil bath at 90° C. at reflux for 3 days. The reaction was quenched by the cautious dropwise addition of acetic acid (100 ml) until the evolution of hydrogen ceased. The stirred reaction mixture was cautiously treated with acetic anhydride solution (500 ml) at such a rate as to cause gentle reflux. The flask was equipped for distillation and stirred and then heating at 90° C. (oil bath temperature) to distil out the tetrahydrofuran. A further portion of acetic anhydride (300 ml) was added, the reaction returned to reflux configuration and stirred and heated in an oil bath at 140° C. for 5 h. The reaction was allowed to cool and filtered. The aluminium oxide precipitate was washed with ethyl acetate and the combined filtrates concentrated on a rotary evaporator at a water bath temperature of 50° C. in vacuo (5 mmHg) to afford an oil. The oil was taken up in ethyl acetate (500 ml) and washed with saturated aqueous potassium carbonate solution. The ethyl acetate solution was separated, dried over sodium sulphate, and concentrated in vacuo to afford an oil. The oil was Kugelrohr distilled in high vacuum to give tris(2-acetoxyethyl)methane (45.3 g, 96%) as an oil. Bp. 220° C. at 0.1 mmHg.

NMR ¹H(CDCl₃), δ 1.66(7H, m, 3×CH₂, CH), 2.08(1H, s, 3×CH₃); 4.1(6H, t, 3×CH₂O).

NMR ¹³C(CDCl₃), δ 20.9, CH₃; 29.34, CH; 32.17, CH₂; 62.15, CH₂O; 171, CO.

### Step 1(d): Removal of Acetate Groups from the Triacetate.

Tris(2-acetoxyethyl)methane (45.3 g, 165 mMn in methanol (200 ml) and 880 ammonia (100 ml) was heated on an oil bath at 80° C. for 2 days. The reaction was treated with a further portion of 880 ammonia (50 ml) and heated at 80° C. in an oil bath for 24 h. A further portion of 880 ammonia (50 ml) was added and the reaction heated at 80° C. for 24 h. The reaction was then concentrated in vacuo to remove all solvents to give an oil. This was taken up into 880 ammonia (150 ml) and heated at 80° C. for 24 h. The reaction was then concentrated in vacuo to remove all solvents to give an oil. Kugelrohr distillation gave acetamide bp 170-180 0.2 mm. The bulbs containing the acetamide were washed clean and the distillation continued. Tris(2-hydroxyethyl)methane (22.53 g, 92%) distilled at bp 220° C. 0.2 mm.

NMR ¹H(CDCl₃), δ 1.45(6H, q, 3×CH₂), 2.2(1H, quintet, CH); 3.7(6H, t 3×CH₂OH); 5.5(3H, brs, 3×OH).

NMR ¹³C(CDCl₃), δ 22.13, CH; 33.95, 3×CH₂; 57.8, 3×CH₂OH.

### Step 1(e): Conversion of the Triol to the tris(methanesulphonate).

To an stirred ice-cooled solution of tris(2-hydroxyethyl)methane (10 g, 0.0676 mol) in dichloromethane (50 ml) was slowly dripped a solution of methanesulphonyl chloride (40 g, 0.349 mol) in dichloromethane (50 ml) under nitrogen at such a rate that the temperature did not rise above 15° C. Pyridine (21.4 g, 0.27 mol, 4 eq) dissolved in dichloromethane (50 ml) was then added drop-wise at such a rate that the temperature did not rise above 15° C., exothermic reaction. The reaction was left to stir at room temperature for 24 h and then treated with 5N hydrochloric acid solution (80 ml) and the layers separated. The aqueous layer was extracted with further dichloromethane (50 ml) and the organic extracts combined, dried over sodium sulphate, filtered and concentrated in vacuo to give tris[2-(methylsulphonyloxy)ethyl]methane contaminated with excess methanesulphonyl chloride. The theoretical yield was 25.8 g.

NMR ¹H(CDCl₃), δ 4.3 (6H, t, 2×CH₂), 3.0 (9H, s, 3×CH₃),2 (1H, hextet, CH), 1.85 (6H, q, 3×CH₂).

### Step 1(f): Preparation of 1,1,1-tris(2-azidoethyl)methane.

A stirred solution of tris[2-(methylsulphonyloxy)ethyl]methane [from Step 1(e), contaminated with excess methylsulphonyl chloride] (25.8 g, 67 mmol, theoretical) in dry DMF (250 ml) under nitrogen was treated with sodium azide (30.7 g, 0.47 mol) portion-wise over 15 minutes. An exotherm was observed and the reaction was cooled on an ice bath. After 30 minutes, the reaction mixture was heated on an oil bath at 50° C. for 24 h. The reaction became brown in colour. The reaction was allowed to cool, treated with dilute potassium carbonate solution (200 ml) and extracted three times with 40/60 petrol ether/diethylether 10:1 (3×150 ml). The organic extracts were washed with water (2×150 ml), dried over sodium sulphate and filtered. Ethanol (200 ml) was added to the petrol/ether solution to keep the triazide in solution and the volume reduced in vacuo to no less than 200 ml. Ethanol (200 ml) was added and reconcentrated in vacuo to remove the last traces of petrol leaving no less than 200 ml of ethanolic solution. The ethanol solution of triazide was used directly in Step 1(g).

CARE: DO NOT REMOVE ALL THE SOLVENT AS THE AZIDE IS POTENTIALLY EXPLOSIVE AND SHOULD BE KEPT IN DILUTE SOLUTION AT ALL TIMES. Less than 0.2 ml of the solution was evaporated in vacuum to remove the ethanol and an NMR run on this small sample:

NMR ¹H(CDCl₃), δ 3.35 (6H, t, 3×CH₂), 1.8 (1H, septet, CH,), 1.6 (6H, q, 3×CH₂).

### Step 1(g): Preparation of 1,1,1-tris(2-aminoethyl)methane.

Tris(2-azidoethyl)methane (15.06 g, 0.0676 mol), (assuming 100% yield from previous reaction) in ethanol (200 ml) was treated with 10% palladium on charcoal (2 g, 50% water) and hydrogenated for 12 h. The reaction vessel was evacuated every 2 hours to remove nitrogen evolved from the reaction and refilled with hydrogen. A sample was taken for NMR analysis to confirm complete conversion of the triazide to the triamine.

Caution: unreduced azide could explode on distillation. The reaction was filtered through a Celite pad to remove the catalyst and concentrated in vacuo to give tris(2-aminoethyl)methane as an oil. This was further purified by Kugelrohr distillation bp. 180-200° C. at 0.4 mm/Hg to give a colourless oil (8.1 g, 82.7% overall yield from the triol).

NMR ¹H(CDCl₃), 2.72 (6H, t, 3×CH₂N), 1.41 (H, septet, CH), 1.39 (6H, q, 3×CH₂).

NMR ¹³C(CDCl₃), δ 39.8 (CH₂NH₂), 38.2 (CH₂.), 31.0 (CH).

### EXAMPLE 2

### Alternative Preparation of 1,1,1-tris(2-aminoethyl)methane

### Step 2(a): Amidation of trimethylester with p-methoxy-benzylamine.

Tris(methyloxycarbonylmethyl)methane [2 g, 8.4 mmol; prepared as in Step 1(b) above] was dissolved inp-methoxy-benzylamine (25 g, 178.6 mmol). The apparatus was set up for distillation and heated to 120° C. for 24 hrs under nitrogen flow. The progress of the reaction was monitored by the amount of methanol collected. The reaction mixture was cooled to ambient temperature and 30 ml of ethyl acetate was added, then the precipitated triamide product stirred for 30 min. The triamide was isolated by filtration and the filter cake washed several times with sufficient amounts of ethyl acetate to remove excess p-methoxy-benzylamine. After drying 4.6 g, 100%, of a white powder was obtained. The highly insoluble product was used directly in the next step without further purification or characterisation.

### Step 2(b): Preparation of 1,1,1-tris[2-(p-methoxybenzylamino)ethyl]methane.

To a 1000 ml 3-necked round bottomed flask cooled in a ice-water bath the triamide from step 2(a) (10 g, 17.89 mmol) is carefully added to 250 ml of 1M borane solution (3.5 g, 244.3 mmol) borane. After complete addition the ice-water bath is removed and the reaction mixture slowly heated to 60° C. The reaction mixture is stirred at 60° C. for 20 hrs. A sample of the reaction mixture (1 ml) was withdrawn, and mixed with 0.5 ml 5N HCl and left standing for 30 min. To the sample 0.5 ml of 50 NaOH was added, followed by 2 ml of water and the solution was stirred until all of the white precipitate dissolved. The solution was extracted with ether (5 ml) and evaporated. The residue was dissolved in acetonitrile at a concentration of 1 mg/ml and analysed by MS. If mono- and diamide (M+H/z=520 and 534) are seen in the MS spectrum, the reaction is not complete. To complete the reaction, a further 100 ml of 1M borane THF solution is added and the reaction mixture stirred for 6 more hrs at 60° C. and a new sample withdrawn following the previous sampling procedure. Further addition of the 1M borane in THF solution is continued as necessary until there is complete conversion to the triamine.

The reaction mixture is cooled to ambient temperature and 5N HCl is slowly added, [CARE: vigorous foam formation occurs!]. HCl was added until no more gas evolution is observed. The mixture was stirred for 30 min and then evaporated. The cake was suspended in aqueous NaOH solution (20-40%; 1:2 w/v) and stirred for 30 minutes. The mixture was then diluted with water (3 volumes). The mixture was then extracted with diethylether (2×150 ml) [CARE: do not use halogenated solvents]. The combined organic phases were then washed with water (lx 200 ml), brine (150 ml) and dried over magnesium sulphate. Yield after evaporation: 7.6 g, 84% as oil.

NMR ¹H(CDCl₃), δ: 1.45, (6H, m, 3×CH₂; 1.54, (1H, septet, CH); 2.60 (6H, t, 3×CH₂N); 3.68 (6H, s, ArCH₂); 3.78 (9H, s, 3×CH₃O); 6.94(6H, d, 6×Ar). 7.20(6H, d, 6×Ar).

NMR¹³C(CDCl₃), δ: 32.17,CH; 34.44, CH₂; 47.00, CH₂; 53.56, ArCH₂; 55.25, CH₃O; 113.78, Ar; 129.29, Ar; 132:61; Ar; 158.60, Ar;

### Step 2(c): Preparation of 1,1,1-tris(2-aminoethyl)methane.

1,1,1-tris[2-p-methoxybenzylamino)ethyl]methane (20.0 gram, 0.036 mol) was dissolved in methanol (100 ml) and Pd(OH)₂ (5.0 gram) was added. The mixture was hydrogenated (3 bar, 100° C., in an autoclave) and stirred for 5 hours. Pd(OH)₂ was added in two more portions (2×5 gram) after 10 and 15 hours respectively. The reaction mixture was filtered and the filtrate was washed with methanol. The combined organic phase was evaporated and the residue was distilled under vacuum (1×10⁻², 110° C.) to give 2.60 gram (50%) of 1,1,1-tris(2-aminoethyl)methane identical with the previously described Example 1.

### EXAMPLE 3

### Preparation of 3-chloro-3-methyl-2-nitrosobutane

A mixture of 2-methylbut-2-ene (147 ml, 1.4 mol) and isoamyl nitrite (156 ml, 1.16 mol) was cooled to −30° C. in a bath of cardice and methanol and vigorously stirred with an overhead air stirrer and treated dropwise with concentrated hydrochloric acid (140 ml, 1.68 mol) at such a rate that the temperature was maintained below −20° C. This requires about 1 h as there is a significant exotherm and care must be taken to prevent overheating. Ethanol (100 ml) was added to reduce the viscosity of the slurry that had formed at the end of the addition and the reaction stirred at −20 to −10° C. for a further 2 h to complete the reaction. The precipitate was collected by filtration under vacuum and washed with 4×30 ml of cold (−20° C.) ethanol and 100 ml of ice cold water, and dried in vacuo to give 3-chloro-3-methyl-2-nitrosobutane as a white solid. The ethanol filtrate and washings were combined and diluted with water (200 ml) and cooled and allowed to stand for 1 h at −10° C. when a further crop of 3-chloro-3-methyl-2-nitrosobutane crystallised out. The precipitate was collected by filtration and washed with the minimum of water and dried in vacuo to give a total yield of 3-chloro-3-methyl-2-nitrosobutane (115 g 0.85 mol, 73%)>98% pure by NMR

NMR ¹H(CDCl₃), As a mixture of isomers (isomer1, 90%) 1.5 d, (2H, CH₃), 1.65 d, (4H, 2×CH₃), 5.85,q, and 5.95,q, together 1H. (isomer2, 10%), 1.76 s, (6H, 2×CH₃), 2.07(3H, CH₃).

### EXAMPLE 4

### Synthesis of bis[N-(1,1-dimethyl-2-N-hydroxyimine propyl)₂-aminoethyl]-(2-aminoethyl)methane (Chelator 1)

To a solution of tis(2-aminoethyl)methane (4.047 g, 27.9 mmol) in dry ethanol (30 ml) was added potassium carbonate anhydrous (7.7 g, 55.8 mmol, 2 eq) at room temperature with vigorous stirring under a nitrogen atmosphere. A solution of 3-chloro-3-methyl-2-nitrosobutane (7.56 g, 55.8 mol, 2 eq) was dissolved in dry ethanol (100 ml) and 75 ml of this solution was dripped slowly into the reaction mixture. The reaction was followed by TLC on silica [plates run in dichloromethane, methanol, concentrated (0.88 sg) ammonia; 100/30/5 and the TLC plate developed by spraying with ninhydrin and heating]. The mono-, di- and tri-alkylated products were seen with RF's increasing in that order. Analytical HPLC was run using RPR reverse phase column in a gradient of 7.5-75% acetonitrile in 3% aqueous ammonia. The reaction was concentrated in vacuo to remove the ethanol and resuspended in water (110 ml). The aqueous slurry was extracted with ether (100 ml) to remove some of the trialkylated compound and lipophilic impurities leaving the mono and desired dialkylated product in the water layer. The aqueous solution was buffered with ammonium acetate (2 eq, 4.3 g, 55.8 mmol) to ensure good chromatography. The aqueous solution was stored at 4° C. overnight before purifying by automated preparative HPLC.

Yield (2.2 g, 6.4 mmol, 23%).

Mass spec; Positive ion 10 V cone voltage. Found: 344; calculated M+H=344.

NMR ¹H(CDCl₃), δ 1.24(6H, s, 2×CH₃), 1.3(6H, s, 2×CH₃), 1.25-1.75(7H, m, 3×CH₂,CH), (3H, s, 2×CH₂), 2.58 (4H, m, CH₂N), 2.88(2H, t CH₂N₂), 5.0 (6H, s, NH₂, 2×NH, 2×OH).

NMR ¹H ((CD₃)₂SO) δ1.1 4×CH; 1.29, 3×CH₂; 2.1 (4H, t, 2×CH₂);

NMR ¹³C((CD₃)₂SO), δ 9.0 (4×CH₃), 25.8 (2×CH₃), 31.0 2×CH₂, 34.6 CH₂,56.8 2×CH₂N, 160.3; C═N.

HPLC conditions: flow rate 8 ml/min using a 25 mm PRP column
A = 3% ammonia solution (sp.gr = 0.88)/water; B = Acetonitrile

| Time | % B |
|---|---|
| 0 | 7.5 |
| 15 | 75.0 |
| 20 | 75.0 |
| 22 | 7.5 |
| 30 | 7.5 |

Load 3 ml of aqueous solution per run, and collect in a time window of 12.5-13.5 min.

### EXAMPLE 5

### Synthesis of Non-radioactive Iodine Barbiturate (Compound 4)

### Step (a): 1-[4-(4-Iodo-phenoxy)phenyl]ethanone.

4-Fluoroacetophenone 25.0 g (181 mmol) was dissolved in DMF (180 ml), then 4-Iodophenol (39.8 g, 181 mmol) and potassium carbonate (30.0 g, 217 mmol) were added. The mixture was refluxed for approximately 7 h, allowed to cool to room temperature and diluted with water. After extraction with methylene chloride or chloroform (3 times), the combined organic phases were washed once with water, and dried (Na₂SO₄). The solvent was removed in vacuo to provide the crude product. The brownish oily residue was recrystallised from hexane/ethyl acetate (7:3) to yield the pure product as a beige crystalline solid, 48.8 g (80%), mp: 99-101° C.

### Step b: Preparation of 2-[4-(4-Iodo-phenoxy)phenyl]-1-morpholin-4-yl-ethanethione.

A mixture of 1-[4-(4-Iodo-phenoxy)phenyl]ethanone (23.0 g, 68.0 mmol), sulphur (5.45 g, 170 mmol) and morpholine (11.8 g, 135 mmol) was heated at 150° C. for 2.5 h. After cooling in an ice bath, the mixture was treated with ethanol for a period of 30-60 min. The precipitated bright yellow solid was collected by suction filtration and recrystallised from ethanol. The product contained a certain amount of sulphur. Yield 26.3 g (88%) of a mustard yellow solid. mp: 123-127° C.

### Step (c): [4-(4-Iodo-phenoxy)phenyl]-acetic acid.

2-[4-(4-Iodo-phenoxy)phenyl]-1-morpholin-4-yl-ethanethione (26.9 g, 61.1 mmol) was heated together with a mixture of glacial acetic acid (54 ml), water (12 ml) and conc. sulphuric acid (8 ml) at 150° C. for 12 h. After cooling to RT, the reaction mixture was diluted with water (ca 10 ml/mmol) and extracted with ethyl acetate (3×). The combined organic extracts were washed with water, dried (Na₂SO₄) and the solvent evaporated in vacuo giving a beige solid (20.1 g, 93°/O). Mp: 148-150° C.

### Step (d): [4-(4-Iodo-phenoxy)-phenyl]-acetic acid methyl ester.

A solution of 17.3 g (48.9 mmol) [4-(4-Iodo-phenoxy)phenyl]-acetic acid in methanol (125 ml) was cooled to −10° C. Thionyl chloride (11.6 g, 7.1 ml, 97.8 mmol) was then added and the reaction mixture heated to reflux for 1 h. After concentration the residue was dissolved in ether. The ether phase was washed with water, dried (Na₂SO₄) and the solvent evaporated to yield a viscous brown-red oil (13.6 g, 76%).

¹H-NMR (300 MHz, CDCl₃, TMS intern): δ [ppm]: 7.49 (d, ³J=8.9 Hz, 2H, H_{Aryl}), 7.15 (d, ³J=8.9 Hz, 2H, H_{Aryl}), 6.84 (d, ³J=8.9 Hz, 2H, H_{Aryl}), 6.66 (d, ³J=8.9 Hz, 2H, H_{Aryl}), 3.59 (s, 3H, CQOCH₃),3.50 (s, 2H, CH₂).

### Step (e): 4-(4-Iodo-phenoxy)phenyl]-malonic acid dimethyl ester.

A suspension of NaH (680 mg, 28.3 mmol) and dimethyl carbonate (8.16 g, 90.6 mmol) in abs. dioxane (70 ml) was heated to 100-120° C., then a solution of [4-(4-Iodo-phenoxy)-phenyl]-acetic acid methyl ester (5.21 g, 14.2 mmol) in abs. dioxane (30 ml) was added dropwise over a period of 1 h. Refluxing was continued for 3 h, then the reaction mixture was allowed to cool to RT overnight. The mixture was poured onto ice water and subsequently extracted with methylene chloride (3×). The combined organic phases were washed with water (1×), brine (1×), dried (Na₂SO₄) and concentrated to give a viscous brown-red oil (5.25 g, 87%).

¹H-NMR (400 MHz, CDCl₃, TMS intern): δ [ppm]: 7.53 (d, ³J=8.7 Hz, 2H, H_{Aryl}), 7.29 (d, ³J=8.7 Hz, 2H, H_{Aryl}), 6.89 (d, ³J=8.7 Hz, 2H, H_{Aryl}), 6.70 (d, ³J=8.7 Hz, 2H, H_{Aryl}), 4.71 (s, 1H, C), 3.68 (s, 6H, COOCH₃).

### Step (f): 5-[4-(4-Iodo-phenoxy)phenyl]-pyrimidine-2,4,6-trione.

Sodium (2 equivalents) was dissolved in ethanol (ca. 10 ml/mg), and urea (1.7 eq.) added to the solution. A solution of 2-[4-(4-Iodo-phenoxy)-phenyl]-malonic acid dimethyl ester (2.22 g, 5.21 mmol) in ethanol was added dropwise, and the reaction mixture heated to reflux for 6 h. After cooling to RT, the mixture was poured onto ice water and adjusted to pH 2, using dilute hydrochloric acid. The precipitate was collected by suction filtration and dried in vacuo giving an amorphous solid. Recrystallisation from methanol/acetonitrile (1:1) gave a brown-yellow solid. Yield 480 mg (22%).

Mp: 285-286° C. (decomposition).

### Step (g): 5-Bromo-5-[4-(4-iodo-phenoxy)phenyl]-pyrimidine-2,4,6-trione.

A suspension of 1.10 g (2.61 mmol) 5-[4-(4-Iodo-phenoxy)phenyl]-pyrimidine-2,4,6-trione, N-bromosuccinimide (557 mg, 3.31 mmol) and a catalytic amount of dibenzoylperoxide (77 mg) in carbon tetrachloride (50 ml) was refluxed for 3 h. After cooling to RT the mixture was concentrated, the residue treated with water and then extracted with ethyl acetate (3×). The combined extracts were washed with brine, dried (Na₂SO₄) and the solvent evaporated giving a viscous brown oil, which was used in the next step without further purification (1.26 g, 96%).

### Step (h): 5-[4-(2-Hydroxyethyl)piperazin-1-yl]-5-[4-(4-iodo-phenoxy)phenyl]-pyrimidine-2,4,6-trione (Compound 4)

A solution of 5-Bromo-5-[4-(4-iodo-phenoxy)phenyl]-pyrimidine-2,4,6-trione (100 mg, 200 μmol) in methanol (5 ml) was treated with N-(2-Hydroxyethyl)piperazine 52.0 mg (400 μmol) and the mixture stirred for 24 h at RT. A precipitate formed after approximately 30-60 min, which was finally collected by suction and dried in vacuo, giving the product as a colourless solid (73.0 mg, 67%).

mp: 255-257° C.

¹H-NMR (300 MHz, DMSO-D₆): δ [ppm]: 11.78 (broad s, 2H), 7.93 (broad, d, ³J=8.9 Hz, 2H, H_{Aryl}), 7.63 (broad, d, ³J=8.9 Hz, 2H, H_{Aryl}), 7.26 (broad, d, ³J=8.9 Hz, 2H, H_{Aryl}), 7.09 (broad, d, ³J=8.9 Hz, 2H, H_{Aryl}), 4.53 (broad, s, 1H, OH), 3.70-3.66 (m, 2H, CH₂), 2.80-2.58 (m, 10H, CH₂).

Compounds 2, ie. 5-[4-(4-bromo-phenoxy)phenyl]-5-[4-(2-hydroxyethyl)piperazin-1-yl]-pyrimidine-2,4,6-trione and Compound 3, ie. 5-[4-(2-hydroxyethyl)piperazin-1-yl]-5-[4 (4-methoxy-phenoxy)phenyl]-pyrimidine-2,4,6-trione were prepared in an analogous manner, by the method of Grams et al [Biol. Chem., 382, 1277-1285 (2001)] starting from Compound 23, ie. 5-bromo-5-[4-(4-bromo-phenoxy)phenyl]-pyrimidine-2,4,6-trione and 5-bromo-5-[4-(4-methoxy-phenoxy)phenyl]-pyrimidine-2,4,6-trione respectively

### EXAMPLE 6

### Synthesis of 5-[4-(2-Hydroxyethyl)piperazin-1-yl]-5-[4-(4-[¹²⁵I]iodo-phenoxy)phenyl]-pyrimidine-2,4,6-trione (Compound 5)

2,5-Dihydroxybenzoic acid (0.6 mg, 3.9 μmol), ascorbic acid (0.8 mg, 4.5 μmol), water for injection (20 μl) and 5 μl (65.3 nmol) CuSO₄5H₂O solution (conc.=3.26 g/l in water for injection) were added to a conical vial containing Compound 2 (50 μl, 209 nmol; conc=2.10 g/l EtOH).

The ice-cooled mixture was degassed for 10 min using a He-flow, then 4 μl [¹²⁵I]NaI in NaOH solution (10.39 MBq) were added and the mixture vortexed. The mixture was heated to 116° C. for 60 min. After cooling to room temperature it was diluted with 50 μl water for injection. The solution was injected to the gradient HPLC-chromatograph with γ- and UV-detector and a Nucleosil 100 C-18 5μ 250×4.6 mm² column with a corresponding 20×4.6 mm² precolumn.

| | | | |
|---|---|---|---|
| HPLC-conditions: | eluent A: | CH₃CN/H₂O/TFA | 950/50/1 |
| | eluent B: | CH₃CN/H₂O/TFA | 50/950/1 |

| | |
|---|---|
| gradient: | eluent B from 92% to 50% |
| | over 45 min, then from 50% to |
| | 92% over 10 min |
| Flow: | 1.5 ml/min |
| λ: | 254 nm |
| Rₜ(product-fraction): | 32.80-33.90 min. |

A part of this fraction (200 μl) was reinjected to the gradient HPLC using the same conditions (see above).

Rₜ(Compound 5): 33.08 min

The quality-control of this product (HPLC, same conditions) did not show any impurities in the γ-channel. In the UV-channel a very slight impurity (31.33 min.) was detected probably caused by the precursor Compound. It is possible to remove the impurity from the fraction with a second HPLC-run.

The Rₜ parameters were established by adding an aliquot of the non-radioactive iodine reference standard (Compound 4) to a second quality-control injection. Radiochemical yield: 20%

### EXAMPLE 7

### 5-[4-(4-Bromo-phenoxy)phenyl]-5-piperazin-1-yl-pyrimidine-2,4,6-trione (Compound 6)

5-Bromo-5-[4-(4-bromo-phenoxy)phenyl]-pyrimidine-2,4,6-trione [Compound 23, Example 5 step (h)] (200 mg, 440 μmol) was dissolved in abs. methanol (5 ml) and treated with piperazine (75.8 mg, 880 μmol). After ca. 10 min a colourless precipitate formed. The reaction mixture was stirred for 24 h at RT., then the precipitate was collected under suction, stirred for 1 h in methanol and dried in vacuo to give 160 mg (79%) of a colourless solid.

mp: 265-266° C. (decomposition).

¹H-NMR (300 MHz, DMSO-D₆): δ [ppm]: 7.34 (broad, d, ³J=8.7 Hz, 2H, H_{Aryl}), 7.22 (broad, d, ³J=8.7 Hz, 2H, H_{Aryl}), 6.82 (broad, d, ³J=8.7 Hz, 2H, H_{Aryl}), 6.79 (broad, d, ³J=8.7 Hz, 2H, H_{Aryl}), 2.55-2.23 (broad, m, 8H, CH₂).

### EXAMPLE 8

### 5-[4-(4-Bromophenoxy)phenyl]-5-[4-(3-fluoropropyl)-piperazin-1-yl)-pyrimidine-2,4,6-trione (Compound 7)

To a solution of Compound 6 (10 mg, 2.2×10⁻⁵ mol) in pyridine (2 ml) under a nitrogen atmosphere at room temperature was added 3-fluoropropyltosylate (1.1 equivalents). The reaction was stirred for 16 hours. The mixture was concentrated and dissolved in methanol (5 ml). The mixture was purified by HPLC (C18, 150×10 mm) and the product eluted after circa 13 minutes. The solvent was removed to give an off-white solid (yield 38%). The structure was confirmed by mass spectral [ES (+ve) 521.1] and ¹H NMR analysis.

### EXAMPLE 9

### Synthesis of the ¹⁸F-Labelled Derivative, Compound 8

### Step (a): Synthesis of 3-[¹⁸F]fluoropropyl tosylate.

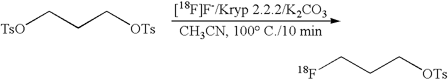

Via a two-way tap Kryptofix 222 (10 mg) in acetonitrile (300 μl) and potassium carbonate (4 mg) in water (300 μl), prepared in a glass vial, was transferred using a plastic syringe (1 ml) into a carbon glass reaction vessel sited in a brass heater. ¹⁸F-fluoride (185-370 MBq) in the target water (0.5-2 ml) was then added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and 1,3-propanediol-di-p-tosylate (5-12 mg) and acetonitrile (1 ml) was added. The pot lid was replaced and the lines capped off with stoppers. The heater was set at 100° C. and labelled at 100° C./10 mins. After labelling, 3-[¹⁸F] fluoropropyl tosylate was isolated by Gilson RP HPLC using the following conditions:

| | |
|---|---|
| Column | u-bondapak C18 7.8 × 300 mm |
| Eluent | Water (pump A): Acetonitrile (pump B) |
| Loop Size | 1 ml |
| Pump speed | 4 ml/min |
| Wavelength | 254 nm |
| Gradient | 5-90% eluent B over 20 min |
| Product Rt | 12 min |

Once isolated, the cut sample (ca. 10 ml) was diluted with water (10 ml) and loaded onto a conditioned C18 sep pak. The sep pak was dried with nitrogen for 15 mins and flushed off with an organic solvent, pyridine (2 ml), acetonitrile (2 ml) or DMF (2 ml). Approx. 99% of the activity was flushed off.

### Step (b): Alkylation of Compound 6.

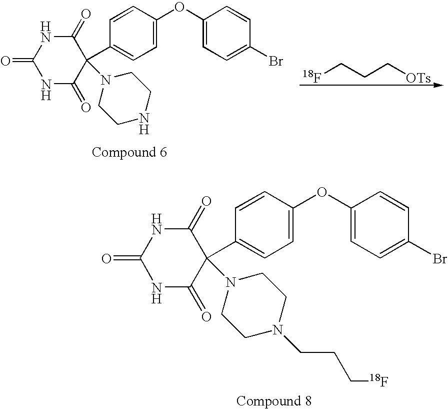

Compound 6 can be alkylated to give Compound 8 by refluxing in pyridine with 3-[¹⁸F] fluoropropyl tosylate.

### EXAMPLE 10

### 5-[4-(4-Bromophenoxy)phenyl]-5-{4-(2-fluoropropylsulfanyl)acetyl]-piperazin-1-yl}-Pyrimidine-2,4,6-trione (Compound 9)

### Step (a): 3-tritllsulfanyl-propan-1-ol [Ph₃CS(CH₂)₃OH].

Triphenylmethanol (390.6 mg, 1.5 mmol) in TFA (10 ml) was added dropwise to a stirred solution of 3-mercaptopropan-1-ol (129.6 μl, 1.5 mmol) in TFA (10 ml). After the addition TFA was evaporated under reduced pressure and the crude product immediately purified by reverse phase preparative chromatography (Phenomenex Luna C18 column, 00G-4253-V0; solvents A=water/0.1% TFA and B=CH₃CN/0.1% TFA; gradient 70-80% B over 60 min; flow 50 ml/minute; detection at 254 nm), affording 372 mg (74%) of pure compound. (analytical HPLC: Vydac C18 column, 218TP54: solvents: A=water/0.1% TFA and B=CH₃CN/0.1% TFA; gradient 70-80% B over 20 min; flow 1.0 ml/minute; retention time 5.4 minutes detected at 214 and 254 nm). Structure verified by NMR.

### Step (b): Methanesulfonic acid 3-tritylsulfanyl-propyl ester [Ph₃CS(CH₂)₃OMs].

To 3-Tritylsulfanyl-propan-1-ol (372.0 mg, 1.11 mmol) dissolved in THF (10 ml) was added triethylamine (151.7 mg, 209 μl, 1.5 mmol) and mesyl chloride (171.9 mg, 116.6 μl, 1.5 mmol). After 1 hour the precipitate was filtered off, THF evaporated under reduced pressure and the crude product purified by reverse phase preparative chromatography Phenomenex Luna C18 column, 00G-4253-V0; solvents A=water/0.1% TFA and B=CH₃CN/0.1% TFA; gradient 80-100% B over 60 min; flow 50 ml/minute; detection at 254 nm), affording 318 mg (69%) of pure compound. (analytical HPLC: Vydac C18 column, 218TP54: solvents: A=water/0.1% TFA and B=CH₃CN/0.1% TFA; gradient 60-70% B over 20 min; flow 1.0 ml/minute; retention time 18.7 minutes detected at 214 and 254 nm). Structure verified by NMR.

### Step (c): (3-fluoropropylsulfanyl)triphenylmethane [Ph₃CS(CH₂)₃F].

Potassium fluoride (1.4 mg, 0.024 mmol) and kryptofix 222 (9.0 mg, 0.024 mmol) were dissolved in acetonitrile (0.2 ml) (heating). Methanesulfonic acid 3-tritylsulfanyl-propyl ester (5 mg, 0.012 mmol) in acetonitrile (0.2 ml) was added. The reaction mixture was heated to 80° C. for 90 minutes. The crude product was purified by reverse phase preparative chromatography (Vydac C18 column, 218TP1022; solvents A=water/0.1% TFA and B=CH₃CN/0.1% TFA; gradient 40-90% B over 40 min; flow 10 ml/minute; detection at 214 nm). A yield of 2.5 mg (62%) of purified material was obtained (analytical HPLC: Phenomenex Luna C18 column, 00B4251-E0: solvents: A=water/0.1% TFA and B=CH₃CN/0.1% TFA; gradient 40-80% B over 10 min; flow 2.0 ml/minute; retention time 8.2 minutes detected at 214 and 254 nm). Structure verified by NMR.

### Step (d): 5-[4-(4-Bromophenoxy)phenyl]-5-{4-(2-fluoropropylsulfanyl)acetyl-piperazin-1-yl}-pyrimidine-2,4,6-trione (Compound 9)

3-Fluoro-tritylsulfanyl-propane (4.1 mg, 0.021 mmol) was stirred with TFA (100 μl), triisopropylsilane (10 μl) and water (10 μl). Water (300 μl) was added followed by 200 μl potassium carbonate (aq). Compound 11 (3.25 mg, 0.0061 mmol) in CH₃CN (500 μl) was added. The pH was adjusted to 10 with potassium carbonate (aq). The mixture was heated to 75° C. for half an hour. The crude product was purified by reverse phase preparative chromatography (Phenomenex Luna C18 column, 00G-4253-N0; solvents A=water/0.1% TFA and B=CH₃CN/0.1% TFA; gradient 20-70% B over 30 min; flow 5 ml/minute; detection at 254 nm). A yield of 2 mg (55%) of purified material was obtained (analytical HPLC: Vydac C18 column, 218TP54: solvents: A=water/0.1% TFA and B=CH₃CN/0.1% TFA; gradient 20-70% B over 20 min; flow 1.0 ml/minute; retention time 17.4 minutes detected at 214 and 254 nm).

¹H NMR (CHCl₃-d1, TMS reference): δ 2.01 (m, 2H), δ 2.72 (broad t, 2H), δ 2.75 (t, 2H), δ 2.79 (broad t, 2H), δ 3.30 (s, 2H), δ 3.30 (broad t, 2H), δ 3.51 (s, 2H), δ 3.64 (broad t, 2H), δ 4.53 (dt, 2H), δ 6.93 (complex d, 2H), δ 6.99 (complex d, 2H), δ 6.93 (complex d, 2H), δ 7.46 (complex d, 2H), δ 7.48 (complex d, 2H), δ 7.77 (complex d, 2H).

### EXAMPLE 11

### 5-[4-(4-Bromophenoxy)-phenyl]-5-{4-(2-[¹⁸F]-fluoropropylsulfanyl)-acetyl]-piperazin-1-yl}-pyrimidine-2,4,6-trione (Compound 10)

### Step (a): Preparation of 3-[¹⁸F] fluoro-tritylsulfanyl-propane.

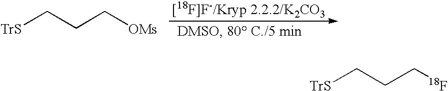

Via a two-way tap Kryptofix 222 (10 mg) in acetonitrile (800 μl) and potassium carbonate (1 mg) in water (50 μl), prepared in a glass vial, was transferred using a plastic syringe (1 ml) to the carbon glass reaction vessel situated in the brass heater. ¹⁸F-fluoride (185-370 MBq) in the target water (0.5-2 ml) was then also added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and trimethyl-(3-tritylsulfanyl-propoxy)silane (1-2 mg) and DMSO (0.2 ml) was added. The pot lid was replaced and the lines capped off with stoppers. The heater was set at 80° C. and labelled at 80° C./5 mins. After labelling, the reaction mixture was analysed by RP HPLC using the following HPLC conditions:

| Column | u-bondapak C18 7.8 × 300 mm | |
|---|---|---|
| Eluent | 0.1% TFA/Water (pump A): 0.1% TFA/Acetonitrile | |
| | (pump B) | |
| Loop Size | 100 ul | |
| Pump speed | 4 ml/min | |
| Wavelength | 254 nm | |
| Gradient | 1 mins | 40% B |
| | 15 mins | 40-80% B |
| | 5 mins | 80% B |

The reaction mixture was diluted with DMSO/water (1:1 v/v, 0.15 ml) and loaded onto a conditioned t-C18 sep-pak. The cartridge was washed with water (10 ml), dried with nitrogen and 3-[¹⁸F] fluoro-1-tritylsulfanyl-propane was eluted with 4 aliquots of acetonitrile (0.5 ml per aliquot).

### Step (b): Preparation of 3-[¹⁸F] fluoro-propane-1-thiol

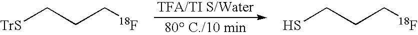

A solution of 3-[¹⁸F] fluoro-1-tritylsulfanyl-propane in acetonitrile (1-2 ml) was evaporated to dryness using a stream of nitrogen at 100° C./10 mins. A mixture of TFA (0.05 ml), triisopropylsilane (0.01 ml) and water (0.01 ml) was added followed by heating at 80° C./10 mins to produce 3-[¹⁸F] fluoro-propane-1-thiol.

### Step (c): Reaction with Compound 11.

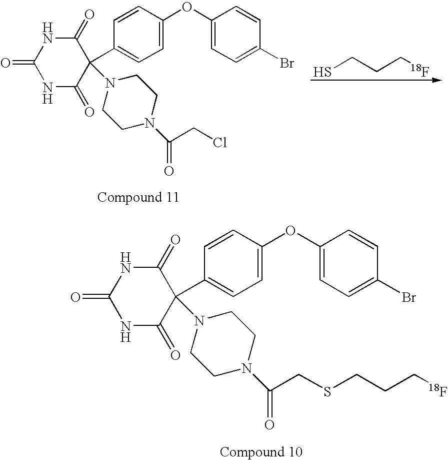

A general procedure for labelling a chloroacetyl precursor is to cool the reaction vessel containing the 3-[¹⁸F] fluoro-1-mercapto-propane from Step (b) with compressed air, and then to add ammonia (27% in water, 0.1 ml) and the Compound 11 precursor (1 mg) in water (0.05 ml). The mixture is heated at 80° C./10 mins.

### EXAMPLE 12

### 5-[4-(4-Bromophenoxy)-phenyl]-5-[4-(2-chloroacetyl)-piperazin-1-yl)-pyrimidine-2,4,6-trione (Compound 11)

To a flask charged with nitrogen and Compound 6 (50 mg, 1.1×10⁻⁴ mol) was added dichloromethane (15 ml). The reaction mixture was cooled in an ice/water bath. Chloroacetyl chloride (14 μl) and triethylamine (14 μl) were added sequentially. The ice bath was removed after 10 minutes and the mixture was allowed to warm to ambient temperature. After 18 hours the sample was concentrated. Methanol (2 ml) was added and the mixture was separated by HPLC (C18, 150×10 mm). The product eluted at 17.5 minutes (52% yield). The structure was confirmed by mass spectral [ES(−ve) 535.1] and ¹H NMR analysis

### EXAMPLE 13

### 3-(4-{5-[4-(4-Bromophenoxy)-phenyl]-2,4,6-trioxohexahydropyrmidin-5-yl}-piperazin-1-yl)N-{5-(2-hydroxylimino-1,1-dimethylpropylamino)-3-[2-(2-hydroxylamino-1,1-dimethylpropylamino)-ethyl]-pentyl}-propionamide (Compound 16).

### Step (a): 5-[4-(4-Bromo-phenoxy)-phenyl]-5-[4-(2-carboxyethyl)piperazin-1-yl]-pyrimidine-2,4,6-trione (Compound 14)

200 mg (440 μmol) 5-Bromo-5-[4-(4-bromo-phenoxy)phenyl]-pyrimidine-2,4,6-trione (Compound 23, Example 5) was dissolved in 2 ml abs. methanol and treated with 83.5 mg (5.28 mmol, 1.2 eq.) 3-(piperazin-lyl)propionic acid. The reaction mixture was heated to reflux for 6 h and then concentrated. The yellow, solid residue was recrystallised from water to give 170 mg (320 μmol, 72%) of a colourless amorphous solid.

mp: 208-210° C. (decomposition).

¹H-NMR (300 MHz, DMSO-D₆): δ [ppm]: 7.64 (broad, d, ³J=8.6 Hz, 2H, H_{Aryl}), 7.50 (broad, d, ³J=8.6 Hz, 2H, H_{Aryl}), 7.14 (broad, d, ³J=8.6 Hz, 2H, H_{Aryl}), 7.10 (broad, d, ³J=8.6 Hz, 2H, H_{Aryl}), 2.75-2.39 (m, 12H, CH₂).

### Step (b)

To a solution of Compound 14 (53 mg) in N,N-dimethylformamide (10 ml) under a nitrogen atmosphere was added TBTU (85 mg) and N-methylmorpholine (0.01 ml) sequentially. After ten minutes Chelator 1 (35 mg) was added and the reaction mixture was stirred at room temperature for 24 hours. The solvent was removed at reduced pressure and the mixture was dissolved in methanol (5 ml). The crude mixture was separated by HPLC. The product eluted after circa 10 minutes (75% yield). The structure was confirmed by mass spectral [ES(+ve) 858.1] and ¹H NMR analysis.

### EXAMPLE 14

### Synthesis of Compound 17

### Step (a): 5-[4-(2-Aminoethyl) piperazin-1-yl]-5-[4-(4-bromo-phenoxy)phenyl]pyrimidine-2,4,6-trione (Compound 12)

200 mg (440 μmol) 5-Bromo-5-[4-(4-bromo-phenoxy)phenyl]pyrimidine-2,4,6-trione (Compound 23, Example 5) was dissolved in 2 ml abs. methanol and treated with 125 mg (127 μl, 9.67 μmol) N-(2-aminoethyl)piperazine. The reaction mixture was stirred at RT, and after ca. 30 min a colourless precipitate formed. Stirring was continued for 16 h, then the precipitate was collected by suction and dried in vacuo to give 100 mg (200 μmol, 45%) of a colourless solid.

mp: 220-223° C. (decomposition).

¹H-NMR (300 MHz, DMSO-D₆): δ [ppm]: 7.67 (broad, d, ³J=9.0 Hz, 2H, H_{Aryl}, ortho to C—Br), 7.55 (broad, d, ³J=9.0 Hz, 2H, H_{Aryl}, ortho to C_{quart.} attached to Pyr.-C 5), 7.15 (broad, d, ³J=9.0 Hz, 2H, H_{Aryl}, meta to C_{quart.} attached to Pyr.-C 5), 7.12 (broad, d, ³J=9.0 Hz, 2H, H_{Aryl}, meta to C—Br), 2.89-2.79 (m, 2H, CH₂—NH₂),2.77-2.65 (m, 4H, N 1-CH₂), 2.39-2.58 (m, 6H, N 4-CH₂).

### Step (b): 4-[2-(4-{5-[4-(4-Bromophenoxy)phenyl]-2,4,6-trioxohexahydropyrimidin-5-yl}-piperazin-1-yl)-ethylcarbamoyl]-butyric acid (Compound 15)

To a solution of Compound 12 in N,N-dimethylformamide (30 ml) under a nitrogen atmosphere was added glutaric anhydride (11 mg) and triethylamine (0.01 ml) sequentially. After 24 hours the solvent was removed under reduced pressure. The crude mixture was dissolved in methanol (5 ml) and separated by HPLC. The product eluted after 12 minutes (50% yield). The structure was confirmed by mass spectral [ES(+ve) 617.9] and ¹H NMR analysis.

### Step (c): Conjugation of 4-[2-(4-{5-[4-(4-bromophenoxy)-phenyl]-2,4,6-trioxohexahydropyrimidin-5-yl}piperazin-1-yl)-ethylcarbamoyl]-butyric acid with Chelator 1

To a solution of Compound 15 (11 mg) in dichloromethane (5 ml) was added TBTU (8 mg) and N-methylmorpholine (0.1 ml) under a nitrogen atmosphere. After 5 minutes, Chelator 1 (6 mg) was added and the mixture stirred for 24 hours. The solvent was removed at reduced pressure and the mixture was dissolved in methanol (5 ml). The mixture was separated by HPLC and the product eluted after circa 10 minutes (58% yield). The structure was confirmed by mass spectral [ES(+ve) 943.2] and ¹H NMR analysis.

### EXAMPLE 15

### 5-[4-2-Hydroxyethyl)piperazin-1-yl]-5-[4-(4-tributylstannylphenoxy)-phenyl]-pyrimidine-2,4,6-trione (Compound 18)

To a suspension of Compound 2 (80 mg) in toluene under a nitrogen atmosphere was added Pd(PPh₃)₄ (200 mg) and hexabutylditin (0.2 ml). The yellow mixture was heated at reflux for 24 hours. After this time the reaction mixture had become black in colour. The reaction mixture was filtered and the solvent was removed at reduced pressure. The crude mixture was dissolved in methanol and purified by HPLC (yield 45%). The structure was confirmed by mass spectral [ES(+ve) 715.1] and ¹H NMR analysis.

### EXAMPLE 16

### 5-[4-(2-Bromoethyl)piperazin-1-yl]-5-[4-(4-bromo-phenoxy)phenyl]-pyrimidine-2,4,6-trione (Compound 13)

To a suspension of Compound 2 (1.40 g, 2.78 mmol) in 80 ml acetonitrile was added 1.46 g (5.56 mmol) triphenylphosphine and 1.84 g (5.56 mmol) carbon tetrabromide. The mixture was heated to reflux for a period of 18 h, cooled to RT and stored at −30° C. overnight. The solid precipitate, which formed upon cooling was collected by suction to give 920 mg (58%) of a beige solid.

¹H-NMR (300 MHz, DMSO-D6): δ [ppm]: 7.56 (d, ³J=9.0 Hz, 2H, HAryl), 7.40 (d, ³J=8.7 Hz, 2H, HAryl), 7.09 (d, ³J=9.0 Hz, 2H, HAryl), 7.02 (d, ³J=8.7 Hz, 2H, HAryl), 3.83-2.70 (m, 12H, CH₂).

### EXAMPLE 17

### Synthesis of Phenyl-Piperazine Derivatives (Compounds 19 to 22)

### (a) General Procedure: Compounds 19 to 21

The corresponding phenyl piperazine (2.0 eq.) was added in portions to a solution of Compound 23 [Example 5 step (h)] (1.0 eq.) in abs. methanol (ca. 2-3 ml/mmol). The reaction mixture was stirred at RT for 20 h. The precipitate was collected under suction and washed with methanol.

In this manner were prepared:

5-[4-(4-Bromo-phenoxy)-phenyl]-5-[4-(4-nitrophenyl)piperazin-1-yl]-pyrimidine-2,4,6-trione (Compound 19)—from the reaction of 400 mg (880 μmol) Compound 2 and 365 mg (1.76 mmol) 1-(4-nitrophenyl)piperazine in 4 ml methanol was obtained 320 mg (63%) of the bright yellow reaction product after 20 h.

¹H-NMR (400 MHz, DMSO-D₆): δ [ppm]:8.22-7.04 (m, 12H, H_{Aryl}), 3.80-2.77 (m, 8H, CH₂).

5-[4-(4-Bromo-phenoxy)phenyl]-5-[4-(4-fluorophenyl)piperazin-1-yl]-pyrimidine-2,4,6-trione (Compound 20)—from the reaction of 400 mg (880 μmol) Compound 2 and 317 mg (1.76 mmol) 1-(4-fluorophenyl)piperazine in 2.5 ml methanol was obtained after recrystallisation from chloroform 290 mg (60%) of the colourless reaction product,

mp: 247-249.5° C.

¹H-NMR (400 MHz, DMSO-d₆): δ [ppm]: 11.66 (s, 2H, NH), 7.59-6.92 (m, 12H, H_{Aryl}), 3.33-2.74 (m, 8H, CH₂).

5-[4-(4-Bromo-phenoxy)-phenyl]-5-[4-(4-trimethylsilyl-phenyl)piperazin-1-yl]-pyrimidine-2,4,6-trione (Compound 21)—from the reaction of 400 mg (880 μmol) Compound 2 and 413 mg (1.76 mmol) 1-(4-trimethylsilylphenyl)piperazine in 2.5 ml methanol was obtained 440 mg (82%) of the colourless reaction product.

mp: 205-210° C.

¹H-NMR (400 MHz, DMSO-D₆): δ [ppm]:7.93-6.77 (m, 12H, H_{Aryl}), 3.64-2.66 (m, 8H, CH₂), 0.20 (s, 9H, SiCH₃).

### (b) 5-[4-(4-Bromo-phenoxy)-phenyl]-5-[4-(4-iodophenyl)piperazin-1-yl]-pyrimidine-2,4,6-trione (Compound 22)

To a suspension of 280 mg (460 μmol) Compound 21 in 25 ml methanol was added a solution of 300 mg (1.84 mmol) iodine monochloride in 5 ml methanol over 40 min at −70° C. under an argon atmosphere. The orange solution was allowed to warm to RT over a period of 1.5 h, diluted with dichloromethane and washed until colourless with 10% aqueous sodium thiosulfate. The water phase was extracted with dichloromethane (3×), washed with brine and dried (Na₂SO₄). The solvent was evaporated and the residue dried in vacuo to give 230 mg of the raw product.

Recrystallisation from methanol gave 62 mg (20%) of the colourless crystalline product.

mp: 210-211° C.

¹H-NMR (300 MHz, DMSO-d₆): δ [ppm]: 11.55 (s, 2H, NH), 7.50-6.63 (m, 12H, H_{Aryl}), 3.03 (s, 4H, CH₂), 2.63 (s, 4H, CH₂).

### EXAMPLE 18

### 5-[4-(4-Bromophenoxy)-phenyl]-5-(4-iodophenylamino)-pyrimidine-2,4,6-trione (Compound 24)

To a solution of Compound 23 (Example 5, 90 mg) in dichloromethane (20 ml) was added 1.1 equivalents of 4-iodoaniline (50 mg) and triethylamine (0.2 ml). The reaction was stirred under a nitrogen atmosphere for 16 hours. The solvent was removed at reduced pressure. The residue was dissolved in methanol (2 ml). The crude mixture was separated by HPLC and the new compound eluted after circa 20.5 minutes. The solvent was removed at reduced pressure to yield an off-white solid (85% yield). The structure was confirmed by mass spectral [ES(−ve) 591.9] and ¹H NMR analysis.

### EXAMPLE 19

### In Vitro Metalloproteinase Inhibition Assay

Compounds 2 to 4 and 20 were studied by the method of Huang W. et al. [J Biol Chem. 272, 22086-2209.1 (1997)].

Thus, a constant concentration of the fluorogenic substrate (1 μM) and the respective MMPs (1 nM) were incubated with increasing amounts of the MMP-inhibitors (1000 pM-100 μM) to determine their IC₅₀ values. The results are shown in Table 1:

TABLE 1

| Compound | MMP-2 IC₅₀ (nM) | MMP-9 IC₅₀ (nM) |
|---|---|---|
| 2 (prior art) | 9 | 4 |
| 3 (prior art) | 9 | 6 |
| 4 | 7 | 2 |
| 20 | 12 | 19 |

### EXAMPLE 20

### Additional In Vitro Metalloproteinase Inhibition Assay

Compounds were screened using the following commercially available Biomol assay kits:

MMP-2 colourimetric assay kit—Catalogue number AK-408,

MMP-9 colourimetric assay kit—Catalogue number AK-410,

MMP-12 colourimetric assay kit—Catalogue number AK-402,

Which are available from Affiniti Research Products Ltd. (Palatine House, Matford Court, Exeter, EX2 8NL, UK).

(a) Test Compound Preparation.

Inhibitors were provided in powdered form, and stored at 4° C. For each inhibitor a 1 mM stock solution in DMSO was prepared, dispensed into 20 μl aliquots and these aliquots stored at −20° C. The stock solution was diluted to give 8 inhibitor concentrations (recommended: 500 μM, 5 μM, 500 nM, 50 nM, 5 nM, 500 pM, 50 pM and 5 pM). Dilutions were made in the kit assay buffer. A five-fold dilution of the inhibitor stocks is made on addition to the assay wells, therefore final concentration range is from 10 μM to 1 pM.

(b) Experimental Procedure.

Details are provided with the commercial kit, but can be summarised as follows:

Prepare test compound dilutions as above,

Add assay buffer to plate,

Add test compounds to plate

Prepare standard kit inhibitor NNGH (see kit for dilution factor)

Add NNGH to control inhibitor wells

Prepare MMP enzyme (see kit for dilution factor)

Add MMP to plate

Incubate plate at 37° C. for ˜15 min

Prepare thiopeptolide substrate (see kit for dilution factor)

Add substrate to plate

Count every 2 min for 1 hr, 37° C., 414 nm on a Labsystems iEMS plate reader.

(c) Results.

The results are given in Table 2:

TABLE 2

| | MMP-2 (Ki) | MMP-9 (Ki) | MMP-12 (Ki) |
|---|---|---|---|
| Compound | (nM) | (nM) | (nM) |
| 7 | 11 | 2 | — |
| 9 | 5 | 0.3 | 11 |
| 16 | 14 | 3 | — |
| 17 | 29 | 10 | 157 |
| 24 | 45 | 20 | — |

### EXAMPLE 21

### ^{99m}Tc-Radiolabelling of Compounds 16 and 17

The ^{99m}Tc complexes were prepared in the same manner, by adding the following to an nitrogen-purged P46 vial:
1 ml N₂ purged MeOH,
100 μg of Compound 16 (or 17) in 100 μl MeOH,
0.5 ml Na₂CO₃/NaHCO₃ buffer (pH 9.2),
0.5 ml TcO₄⁻ from Tc generator,
0.1 ml SnCl₂/MDP solution,
(solution containing 10.2 mg SnCl₂ and 101 mg methylenediphosphonic acid in 100 ml N₂ purged saline).

For ^{99m}Tc-Compound 16 the activity of solution was measured to be 216 MBq and the solution was heated to 37° C. for 33 min. An ITLC (Instant thin layer chromatography) using SG plates and a mobile phase of MeOH/(NH₄OAc 0.1M) 1:1 showed 1% RHT (reduced hydrolysed Tc) at the origin. HPLC analysis showed 93% of ^{99m}Tc-Compound 16 to give an RCP of 92%.

^{99m}Tc-Compound 17 was prepared in a similar manner. The activity of the complex solution was measured as 203 MBq. ITLC gave 4% colloid and HPLC analysis showed 93% ^{99m}Tc-Compound 17 to give an RCP of 89%.

HPLC analyses were carried out using an Xterra RP18, 3.5 μm, 4.6×150 mm column using an aqueous mobile phase (solvent A) of 0.06% NH₄OH and organic mobile phase (solvent B) of acetonitrile and a flow rate of 1 ml/min. Typical gradients used were as follows: 0-5 min (10-30% B), 5-17 min (30% B), 17-18 min (30-100% B), 18-22 min (100% B) and 22-24 min (100-10% B). The retention time of ^{99m}Tc-Compound 16 was 7.6 min while that of ^{99m}Tc-Compound 17 was 7.5 min.

### EXAMPLE 22

### General Procedure for Electrophilic Radioidination of Barbiturate Precursors

10 μL of freshly prepared 0.01M peracetic acid in water (1×10⁻⁷ mol) is added to a silanised vial containing the precursor substrate (1×10⁻⁷ mol) in an appropriate solvent, together with 200 μL 0.2M NH₄OAc buffer (pH=4), 100 μL Na¹²⁷I (1×10⁻⁷ mol) and Na¹²³I. The reaction is agitated gently and the product purified by HPLC.

What is claimed is:

1. An imaging agent which comprises a synthetic barbituric acid matrix metalloproteinase inhibitor labelled at the 5-position of the barbituric acid with an imaging moiety, wherein the imaging moiety can be detected following administration of said labelled synthetic barbituric acid matrix metalloproteinase inhibitor to the mammalian body in vivo, and said imaging moiety is chosen from:
(i) a radioactive metal ion;
(ii) a paramagnetic metal ion;
(iii) a gamma-emitting radioactive halogen;
(iv) a positron-emitting radioactive non-metal;
(v) a hyperpolarised NMR-active nucleus;
(vi) a reporter suitable for in vivo optical imaging;
(vii) a β-emitter suitable for intravascular detection.

2. The imaging agent of claim 1, where the synthetic barbituric acid matrix metalloproteinase inhibitor ligand conjugate is of Formula I:
[{inhibitor}-(A)ₙ]ₘ-[imaging moiety] (I)
where:
{inhibitor} is the synthetic barbituric acid matrix metalloproteinase inhibitor;
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid or a monodisperse polyethyleneglycol (PEG) building block;
R is independently chosen from H, C₁₋₄ alkyl, C₂₋₄ alkenyl, C₂₋₄ alkynyl, C₁₋₄ alkoxyalkyl or C₁₋₄ hydroxyalkyl;
n is an integer of value 0 to 10; and
m is 1, 2 or 3.

3. The imaging agent of claim 1, where the synthetic barbituric acid matrix metalloproteinase inhibitor is conjugated to a ligand, and said ligand forms a metal complex with the radioactive metal ion or paramagnetic metal ion.

4. The imaging agent of claim 3, where the ligand is a chelating agent.

5. The imaging agent of claim 3, where the radioactive metal ion is a gamma emitter or a positron emitter.

6. The imaging agent of claim 5, where the radioactive metal ion is ^{99m}Tc, ¹¹¹In, ⁶⁴Cu, ⁶⁷Cu, ⁶⁷Ga or ⁶⁸Ga.

7. The imaging agent of claim 1 where the gamma-emitting radioactive halogen imaging moiety is ¹²³I.

8. The imaging agent of claim 1, where the positron-emitting radioactive non-metal is chosen from ¹⁸F, ¹¹C or ¹³N.

9. The imaging agent of claim 1 where the synthetic 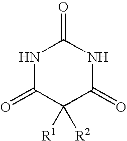
barbituric acid matrix metalloproteinase inhibitor is of Formula IV:
(IV)
where:
R¹ is R″ or a Z group;
R² is R″, Y or —NR⁴R⁵, where R⁴ is H or an R″ group, R⁵ is H, C₂₋₁₄ acyl, C₂₋₁₀ aminoalkyl or (N—C₂₋₁₄ acyl)C₂₋₁₀ aminoalkyl or an R″ group, or R⁴ and R⁵ together with the N atom to which they are attached form an optionally (N—C₂₋₁₄)acylated C₂₋₈ cycloaminoalkylene ring;
R″ is independently C₁₋₁₄ alkyl, C₃₋₈ cycloalkyl, C₂₋₁₄ alkenyl, C₁₋₁₄ fluoroalkyl, C₁₋₁₄ perfluoroalkyl, C₆₋₁₄ aryl, C₂₋₁₄ heteroaryl or C₇₋₁₆ alkylaryl;
Z is a group of formula -A¹O[A²O]ₚR³ where p is 0 or 1, and A¹ and A² are independently C₁₋₁₀ alkylene, C₃₋₈ cycloalkylene, C₁₋₁₀ perfluoroalkylene, C₆₋₁₀ arylene or C₂₋₁₀ heteroarylene, and R³ is an R group where R is independently chosen from H, C₁₋₄ alkyl, C₂₋₄ alkenyl, C₂₋₄ alkynyl, C₁₋₄ alkoxyalkyl or C₁₋₄ hydroxyalkyl;
Y is a group of formula: 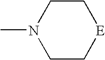
where E is CR₂, O, S or NR⁶; and R⁶ is C₂₋₁₄ acyl, or an R″ or Z group.

10. The imaging agent of claim 9, where R² is Y or —NR⁴R⁵.

11. The imaging agent of claim 9, where the imaging moiety is attached to the R² substituent.

12. The imaging agent of claim 9, of Formula V: 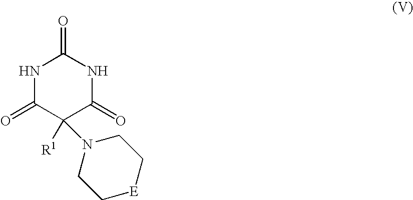
where E is CHR or NR⁶ and R¹ is C₆₋₁₄ n-alkyl, or C₆₋₁₄ aryl.

13. The imaging agent of claim 12, where E is NR⁶ and R⁶ is C₂₋₁₄ acyl; —(CH₂)_{d}OH, where d is 2, 3, 4 or 5; or —C₆H₄X, where X is H, C₁₋₄ alkyl, Hal, OR, NR₂, NO₂ or SO₂NR⁷R⁸, where R⁷ and R⁸ are independently R groups, and R is as defined in claim 9.

14. The imaging agent of claim 12, where R¹ is n-octyl, n-decyl, biphenyl, C₆H₅X or —C₆H₄—O—C₆H₄X where X is as defined in claim 13.

15. A pharmaceutical composition which comprises the imaging agent of claim 1 together with a biocompatible carrier, in a form suitable for mammalian administration.

16. A radiopharmaceutical composition which comprises the imaging agent of claim 1, wherein the imaging moiety is radioactive, together with a biocompatible carrier, in a form suitable for mammalian administration.

17. The radiopharmaceutical composition of claim 16, where the imaging moiety comprises a radioactive metal ion.

18. The radiopharmaceutical composition of claim 16, where the imaging moiety comprises a positron-emitting radioactive non-metal or a gamma-emitting radioactive halogen.

19. A conjugate of a synthetic barbituric acid matrix metlloproteinase inhibitor with a ligand, wherein the barbituric acid comprises a 5-position substituent, and said 5-position substituent comprises a ligand capable of forming a metal complex with a radioactive or paramagnetic metal ion which is resistant to transchelation.

20. The conjugate of claim 19, of Formula Ib:
[{inhibitor}-(A)ₙ]ₘ-[ligand] (Ib),
where {inhibitor}, A, n and m are as defined in claim 2.

21. The conjugate of claim 19, wherein the synthetic barbituric acid matrix metalloproteinase inhibitor is of Formula IV or Formula V of claims 9 to 14.

22. The conjugate of claim 19, wherein the ligand is a chelating agent.

23. The conjugate of claim 22, wherein the chelating agent has a diaminedioxime, N₂S₂, or N₃S donor set.

24. A kit for the preparation of the radiopharmaceutical composition of claim 17, which comprises:
a conjugate of a synthetic barbituric acid matrix metalloproteinase inhibitor with a ligand, wherein the barbituric acid comprises a 5-position substituent, and said 5position substituent comprises a ligand capable of forming a metal complex with a radioactive or paramagnetic metal ion which is resistant to transchelation, said conjugate being of Formula Ib:
[{inhibitor}-(A)ₙ]ₘ-[ligand] (Ib),
where {inhibitor} A, n and m are as defined in claim 2; and
wherein the ligand is a chelating agent.

25. The kit of claim 26, where the radioactive metal ion is ^{99m}Tc, and the kit further comprises a biocompatible reductant.

26. A kit for the preparation of the radiopharmaceutical composition of claim 18, which comprises a precursor in sterile form which is a non-radioactive derivative of the barbituric acid matrix metalloproteinase inhibitor of claims 1, wherein said non-radioactive derivative is capable of reaction with a source of the positron-emitting radioactive non-metal or gamma-emitting radioactive halogen to give the desired radiopharmaceutical.

27. The kit of claim 26, where the source of the positron-emitting radioactive non-metal or gamma-emitting radioactive halogen is chosen from:
(i) halide ion;
(ii) F⁺ or I⁺; or
(iii) an alkylating agent chosen from an alkyl or fluoroalkyl halide, tosylate, triflate or mesylate;
(iv) HS(CH₂)₃¹⁸F.

28. The kit of claim 26, wherein the non-radioactive derivative is chosen from:
(i) an organometallic derivative such as a trialkylstannane or a trialkylsilane;
(ii) a derivative containing an alkyl or aryl iodide or bromide, alkyl tosylate or alkyl mesylate for nucleophilic substitution;
(iii) a derivative containing an aromatic ring activated towards nucleophilic or electrophilic substitution;
(iv) a derivative containing a functional group which undergoes facile alkylation;
(v) a derivative which undergoes alkylation with an alkyl thiol to give a thioether.

29. The kit of claim 26, where the precursor is bound to a solid phase.

30. Use of the imaging agent of claim 1 for the diagnostic imaging of atherosclerosis.

31. Use of the imaging agent of claim 1 for the diagnostic imaging of unstable plaques.

32. Use of the imaging agent of claim 1 for the intravascular detection of atherosclerosis.
